(19) United States
(12) Patent Application Publication
(10) Pub. No.: US 2020/0138318 A1
(43) Pub. Date: May 7, 2020

(54) METHODS AND SYSTEMS FOR SEIZURE ANALYSIS

(71) Applicant: Brain Sentinel, Inc., San Antonio, TX (US)

(72) Inventors: Damon P. Cardenas, San Antonio, TX (US); Jose E. Cavazos, San Antonio, TX (US); Isa Conradsen, Kobenhavn S (DK); Michael R. Girouard, Shavano Park, TX (US); Jonathan J. Halford, Mount Pleasant, SC (US); Luke E. Whitmire, San Antonio, TX (US)

(21) Appl. No.: 16/579,536

(22) Filed: Sep. 23, 2019

Related U.S. Application Data
(63) Continuation-in-part of application No. 14/407,249, filed on Dec. 11, 2014, now Pat. No. 10,420,499.
Application No. 14/407,249 is a continuation-in-part of application No. 16/465,841, filed on May 31, 2019.
Application No. 16/465,841 is a continuation of application No. 15/491,883, filed on Apr. 19, 2017.
Application No. 15/491,883 is a continuation-in-part of application No. 15/491,883, filed on Apr. 19, 2017.
(60) Provisional application No. 62/429,359, filed on Dec. 2, 2016.
(60) Provisional application No. 62/429,359, filed on Dec. 2, 2016.

(30) Foreign Application Priority Data
Jun. 12, 2012 (DK) PA 2012 70323

(51) Int. Cl. A61B 5/04; A61B 5/00; A61B 5/0488

(52) U.S. Cl. CPC A61B 5/04014; A61B 5/4094; A61B 5/726; A61B 5/7264; A61B 5/0488

(57) ABSTRACT

Methods and apparatuses for detecting and characterizing seizures are described. In some embodiments, the methods and apparatuses include collecting an electrical signal from one or more electrodes disposed on the head or scalp of a patient and processing the electrical signal to determine the dynamics of a seizure event.

### CROSS REFERENCE

This application claims priority to U.S. Provisional Application No. 62/746,448, filed Oct. 16, 2018 and titled “Methods and Systems for Seizure Analysis. This application is a continuation-in-part of U.S. application Ser. No. 14/407,249, filed Dec. 11, 2014, and titled “Method and System of Detecting Seizures” which is a national phase entry of International Application No. PCT/DK2013/050189, filed Jun. 11, 2013, and titled “Method and System of Detecting Seizures”, which claims priority to Danish Application PA 201270323, filed Jun. 12, 2012. This application is also a continuation-in-part of U.S. application Ser. No. 16/465,841, filed May 31, 2019 which is a national stage entry of International Application No. PCT/US2017/064377, filed Dec. 2, 2017, and titled “Semiology of Seizures Including Muscle Signals Collected from Electroencephalography Electrodes,” which is a continuation of U.S. application Ser. No. 15/491,883, filed Apr. 19, 2017 and titled “Systems and Methods for Characterization of Seizures,” which claims priority to U.S. Provisional Application No. 62/429,359, filed Dec. 2, 2016. This application is also a continuation-in-part of U.S. application Ser. No. 15/491,883, filed Apr. 19, 2017, and titled “Systems and Methods for Characterization of Seizures,” which claims priority to U.S. Provisional Application No. 62/429,359, filed Dec. 2, 2016. This application claims priority to each of the above references. The disclosures of each of the above references are herein fully incorporated by reference.

### FIELD

The present application relates to systems and methods for detecting seizures and/or characterizing the dynamics of detected seizure activity based on muscle-related electrical signals.

### BACKGROUND

A seizure may be characterized as abnormal or excessive synchronous activity in the brain. At the beginning of a seizure, neurons in the brain may begin to fire at a particular location. As the seizure progresses, this firing of neurons may spread across the brain, and in some cases, many areas of the brain may become engulfed in this activity. Seizure activity in the brain may cause the brain to send electrical signals through the peripheral nervous system activating different muscles of the body.

Seizures may characterize a number of distinct or related disease states. For example, seizures may be identified not only in patients with epilepsy, but also in patients who suffer from other disorders, including disorders characterized by psychogenic non-epileptic seizures (PNES). Notably, it may be particularly difficult to diagnose whether a patient is suffering from epilepsy or another related disorder which may present similar symptoms to epilepsy, such as PNES. Moreover, epilepsy itself may have a number of different root causes, characterization of which may be extremely difficult. Currently, electroencephalography (EEG) monitoring combined with video recordings (video-EEG) is considered the preferred way of identifying whether a patient may be experiencing epilepsy and/or suffering from a related disorder. However, even with video-EEG monitoring, it may sometimes be difficult to diagnose a patient as having epilepsy or another condition.

Electroencephalography techniques generally focus on electrical activity associated with neuronal activation. Where electrical signals related to motor muscle activity are collected together with neuronal signals, such signals are generally considered as unwanted or noise signals. Although not typically done in clinical diagnosis, it may be advantageous to analyze a patient for signals associated with both activation of muscle fibers and neuronal activation. This may be done by collecting an electromyography (EMG) signal using electrodes placed on or near the skin, over a muscle, to detect electrical activity resulting from muscle fiber activation so as to provide valuable information about muscle-related electrical activation during seizures. For example, a study by one of the inventors (see Conradsen et al., Patterns of muscle activation during generalized tonic and tonic-clonic epileptic seizures, Epilepsia, Volume 52, Issue 11, 2011) has shown that the quantitative sEMG (surface electromyography) parameters calculated for the whole seizure period differed significantly among a GTC seizure, a tonic seizure and a voluntary activation acted by healthy controls. Further advances herein address additional deficiencies in previous EMG systems, including, for example, definition of reliable thresholds for different patients and means for differentiating phase boundaries to enhance reliability for analysis of seizure semiology.

To measure signals associated with muscle activation, sensor electrodes may be placed over one or more peripheral muscles, such as the biceps, triceps, or quadriceps. Accordingly, a distinct set of electrodes and associated collection system separate from EEG may be used. However, such systems may not always be available or used, and many clinical diagnoses of seizures involve video-EEG without any attempt to measure muscle activation during seizure events. Accordingly, there remains a need for improved methods of combining EEG with methods for measuring muscle activity, including methods that may not rely on use of an additional set of electrodes or other complicated instrumentation beyond which may be used for EEG signal detection. Systems and methods herein may be used to combine EEG with electromyography (EMG) and may advantageously do so without demanding specialized training or equipment generalized used with EMG data collection.

For at least the above deficiencies, persons suffering from seizures may often be admitted to special clinics or hospitals for diagnosis and treatment of seizure conditions. In specialized facilities the medical staff, such as primary caregivers, doctors or neurologists, may attempt to analyze, manage, and classify seizures, such as based on seizure semiology (also referred to herein as seizure dynamics). Such may, for example, be useful in differentiating different types of seizures and in identifying seizure-related disorder such as PNES that may be treated differently than other seizure disorders, such as epilepsy. However, because previously existing methods of analysis of seizures using only EEG or video EEG may not be sufficient to characterize different types of seizures, seizures are often detected and recorded by using multiple signal processing means, e.g., video-EEG, CT- and MM-scanning, EEG and CT/MRI-scanning, motion detection or other signal processing means. This process is not only very time consuming, but also requires a lot of data analysis either automatically or manually in order to determine the characteristics of the seizure. Moreover, the dynamics or semiology of seizures, including the start-points and stop-points of periods and of the different phases occurring in the seizure period are often determined manually by person(s) specially trained in analyzing the signals. Even with specially trained staff, this may lead to significant uncertainty and lack of reproducibility in determining the precise start- and stop-point for each phase and the length (period) of the seizure. Improved methods of determining the dynamics of seizures, including methods that may increase accuracy and reduce variability over other manual methods would constitute a significant technological advance. Systems and methods herein may be used to provide standardized and more reliable means for detection and characterization of seizures and their dynamics.

In conclusion, there remains a need for improved systems and methods of determining the dynamics of a seizure and properly diagnosing patients who exhibit seizure or seizure-like symptoms. There is a further need for improved systems and methods for determining the dynamics of a seizure and diagnosing patients using existing EEG equipment with or without corroborating video data. There further remain a need for improved methods of recording muscle-related electrical signals and for performing reproducible seizure detection and analysis of seizure dynamics using such signals. There further remain a need for improved methods of recording muscle-related electrical signals and for performing reproducible seizure detection and analysis of seizure dynamics automatically and using methods that may be appropriately used by caregivers responding to seizure events. Methods and systems herein may be directed to technologies for improving diagnosis of seizures and for determining seizure dynamics based on muscle activity, which in some embodiments, may be collected concurrently with EEG, meeting the above and other needs.

### SUMMARY

It is an object of some embodiments of the systems and methods described herein to provide systems and methods capable of analyzing the dynamics or semiology of tonic-clonic seizures. It is a further object of some embodiments of the systems and methods described herein to provide a system and method capable of determining the length of the whole seizure period. It is further an object of some embodiments of the systems and methods described herein to analyze the semiology of seizure events automatically with improved precision, accuracy or both over manual methods. It is further an object of some embodiments of the systems and methods described herein to perform seizure semiology and/or to perform additional analyses of seizure events in clinics and hospitals with various capabilities and resources. For example, in some hospitals or clinics, capabilities may be available to collect muscle-related electrical signals using dedicated EMG electrodes placed over one or more peripheral muscles of a patient. Those muscle-related electrical signals may be used individually or with EEG data to determine the semiology of seizures, identify PNES disorders, perform other analyses, or combinations thereof. In other embodiments, muscle-related electrical signals may be collected from electrodes placed on the head or scalp of a patient, without needing to apply separate electrodes outside of those dedicated for EEG data collection. Those muscle-related electrical signals may be used in place of or in addition to other muscle-related electrical signals and used individually or combination with other data to determine the semiology of seizures, identify PNES disorders, perform other analyses, or combinations thereof.

In some embodiments, systems and methods herein may include collecting muscle-related electrical signals together with electrical activity collected directly from the brain, such as by using EEG electrodes. In other embodiments, muscle-related electrical signals may be collected using electrodes attached to one or more peripheral muscles of a patient. In both embodiments, the systems and methods herein provide robust and cost-effective platforms for seizure detection and characterization which is a distinct technological improvement over existing approaches for seizure detection and analysis, such as those approaches that may rely on multi-modal detection and/or manual analysis of collected data.

In some embodiments, methods and systems for determining the semiology or dynamics of seizures are described. Systems may, for example, include one or more processor units capable of receiving an electrical signal indicative of muscle activation from an electrode disposed on the head or scalp of a patient; a first analysis module configured to analyze at least a portion of the received electrical signal in a first frequency band within a range from about 32 Hz to about 512 Hz to provide a first output signal, and a second analysis module configured to analyze at least a portion of the received electrical signal in a second frequency band within a range from about 2 Hz to about 16 Hz to provide a second output signal; and a time-point extraction module configured to compare at least one of the first output signals or said second output signals to at least one threshold value and to determine a starting point of at least one seizure event phase based on meeting the at least one threshold value.

In some embodiments, methods and systems for determining the semiology or dynamics of seizures are described. Systems may, for example, include one or more processor units capable of receiving an electrical signal indicative of muscle activation from an electrode disposed on a peripheral muscle of a patient; a first analysis module configured to analyze at least a portion of the received electrical signal in a first frequency band within a range from about 32 Hz to about 512 Hz to provide a first output signal, and a second analysis module configured to analyze at least a portion of the received electrical signal in a second frequency band within a range from about 2 Hz to about 16 Hz to provide a second output signal; and a time-point extraction module configured to compare at least one of the first output signals or said second output signals to at least one threshold value and to determine a starting point of at least one seizure event phase based on meeting the at least one threshold value.

In some embodiments, systems herein may include one or more processor units capable of receiving an electrical signal from an electrode disposed on the head or scalp of a patient, the one or more processor units configured to analyze a seizure event based on muscle-related electrical activity; a first analysis module configured to analyze at least a portion of the received electrical signal within a first frequency band associated with tonic-clonic seizure activation of muscle to provide a first output signal, and a second analysis module configured to analyze at least a portion of the received signal within a second frequency band associated with clonic phase seizure activation of muscle to provide a second output signal; and a time-point extraction module configured to compare at least one of the first output signals or said second output signals to at least one threshold value and to determine a starting point of at least one seizure event phase based on meeting the at least one threshold value.

In some embodiments, systems herein may include one or more processor units capable of receiving an electrical signal from an electrode disposed on a peripheral muscle of a patient, the one or more processor units configured to analyze a seizure event based on muscle-related electrical activity; a first analysis module configured to analyze at least a portion of the received electrical signal within a first frequency band associated with tonic-clonic seizure activation of muscle to provide a first output signal, and a second analysis module configured to analyze at least a portion of the received signal within a second frequency band associated with clonic phase seizure activation of muscle to provide a second output signal; and a time-point extraction module configured to compare at least one of the first output signals or said second output signals to at least one threshold value and to determine a starting point of at least one seizure event phase based on meeting the at least one threshold value.

### BRIEF DESCRIPTION OF THE DRAWINGS

FIG. 1 is a diagram of a system for detecting and/or analyzing seizures activity, the system configured for analysis of muscle-related electrical activity.

FIG. 2 is a diagram of a system for detecting and/or analyzing seizure activity, the system including electrodes positioned on the head or scalp of a patient.

FIG. 3 is a diagram of a system for detecting and/or analyzing seizure activity, the system including electrodes positioned on the head or scalp of a patient using a headset or frame.

FIG. 4 is a diagram of a system for detecting and/or analyzing seizure activity, the system including different processors for analyzing different types of signals from electrodes positioned on the head or scalp of a patient.

FIG. 5 is a diagram of a system for detecting and/or analyzing seizure activity, the system including a processor for analyzing different types of signals from electrodes positioned on the head or scalp of a patient.

FIG. 6A shows data for an electrical signal obtained from an electrode.

FIG. 6B shows data for an electrical signal obtained form an electrode showing signal contributions from each of a low frequency band and a high frequency band.

FIG. 7 shows data for a transformed signal for the high frequency band and for the low frequency band.

FIG. 8 shows the ratio of the high and low frequency bands shown in FIG. 7.

FIG. 9 is a diagram of another embodiment of a system for detecting and/or analyzing seizure activity.

FIG. 10 shows data for an average signal of a sensed signal.

FIG. 11 shows data for the median frequencies of a sensed signal.

FIG. 12 shows data for the mean coherence of a sensed signal.

FIG. 13 shows a flowchart of a method for determining the dynamics or semiology of a detected seizure event.

FIG. 14 shows a flowchart of a method for classifying a detected seizure.

FIG. 15 shows the count of zero-crossings of the high frequency band shown in FIG. 7.

### DETAILED DESCRIPTION

The following terms as used herein should be understood to have the indicated meanings.

The term “peripheral muscle” refers to any muscle outside of the head or scalp.

The term “seizure-detection routine” refers to a method or part of a method that may be used to collect or analyze patient data and detect seizure activity or indicate increased risk that a seizure may occur, be occurring, or may have occurred. A seizure-detection routine may be run individually or may be run in combination with other seizure-detection routines or methods. One or more seizure-detection routines may sometimes be used to identify parts of data that may include one or more seizure event. For example, one or more seizure-detection routines may sometimes be used to search for seizure events that may be part of stored or archived data. Further by way of example, one or more seizure-detection routines may sometimes be used to monitor a patient in real-time, such as may be used to initiate one or more alarms based on detection of seizure activity or activity that may indicate increased risk that a seizure may occur, be occurring, or may have occurred.

The term “seizure event” as used herein, unless the context indicates otherwise, includes physiological events wherein a patient has suffered a seizure or exhibited physiological activity resembling seizure activity, even if a true seizure may not have occurred.

Where a range of values is described, it should be understood that intervening values, unless the context clearly dictates otherwise, between the upper and lower limit of that range and any other stated or intervening value in other stated ranges, may be used within embodiments herein.

The systems, apparatuses, and methods described herein may be used for detection and/or analysis of seizure events and may include collecting an electrical signal using one or more electrodes, such as may be part of one or more sensor units. For example, as described in U.S. application Ser. No. 14/407,249, filed Jun. 11, 2013, and titled “Method and System of Detecting Seizures,” which is fully incorporated herein by reference, systems for detecting seizures and/or analyzing the dynamics of a seizure may include one or more electrodes included in one or more sensor units, which may, for example, be disposed on or in the proximity of one or more peripheral muscles of a patient. As further described in this disclosure, one or more electrodes may be disposed on or in the proximity of one or more peripheral muscles of a patient, one or more muscles of the head or scalp of a person, or both, and electrical signals resulting from muscle activity measured therefrom.

In some embodiments, seizure detection and analysis systems herein may comprise one or more components, such as may be embodied in software, hardware, or both, which may be used to modify an existing EEG system. Thus, the one or more components may be applied in use as a convenient add-on or modification to an existing EEG system. Accordingly, such technology may be readily used with available EEG machinery in clinics and hospitals of various types and capabilities, resulting in new functionality over existing or unmodified EEG systems. Notably, some communities, such as those in rural areas, may not have ready access to specialized facilities having specially trained staff and/or equipment suitable for analyzing seizures using multiple processing modalities. And, the systems and methods herein may provide low-cost and effective means for performing seizure analysis that otherwise may not be performed. In some embodiments, the seizure detection and analysis systems herein may operate independent of existing EEG equipment, such as in the form of a stand-alone system configured for collecting electrical signals from one or more peripheral muscles and/or one or more muscles of the head and/or scalp of a patient.

Electrodes positioned on the head or scalp of a patient, such as may be traditionally applied for measurement of electrical activity in the brain, may herein be referred to as EEG electrodes, irrespective of whether they are used for collecting signals used for analysis of muscle-related or non-muscle related signals. The international 10-20 system may be used to describe the positioning of EEG electrodes over the head or scalp of a patient. Using the international 10-20 system, an EEG electrode may be described with a letter, which designates the lobe of the brain present underneath the positioned electrode, and a number, which describes a particular position of the electrode and hemisphere of the brain on which the electrode is positioned. In typical EEG detection systems, muscle-related components of signals are usually considered a contaminant or noise source obscuring the analysis of signals directly manifested in the brain. For example, if detected, muscle-related signals may often be used to identify a contaminant or artifact as may clutter or obscure other collected data. Accordingly, processing suitable for reducing contaminant signals associated with muscle activity is commonly incorporated in many EEG systems. In contrast, in some embodiments herein, muscle-related-electrical activity is detected using EEG electrodes and may be used to successfully characterize seizures or seizure-related events including, for example, the dynamics of a seizure event or other seizure-related characteristics.

In some embodiments, one or more filters are used to process an electrical signal collected from EEG electrodes to remove signal components directly manifested from physiological activity in the brain. For example, in some embodiments, at least a portion of one or more of the delta band, theta band, or both are selectively filtered from an electrical signal. By removing those signal components, other portions of an electrical signal, such as signals related to clonic-discharge bursts or other signals associated with muscle-related electrical activity, may be detected with improved sensitivity and/or selectivity.

In some embodiments, an electrical signal is collected and/or analyzed using one or more EEG electrodes, wherein the one or more EEG electrodes are positioned at one or more of any of the standardized positions designated in the international 10-20 system. For example, an electrode signal may be collected using one or more EEG electrodes, wherein the position(s) of the one or more EEG electrodes may be selected from one or more of the F7, F8, T3, and T4 positions as defined in the international 10-20 system. It may be noted that the aforementioned positions place the EEG electrodes in close proximity to the frontalis and temporalis muscles of the head. In some embodiments, EEG electrodes are specifically placed over the frontalis and temporalis muscles or EEG electrodes are configured in some way to enhance the strength of muscle-related signal components or relative strength of muscle-related signals versus other signal components. For example, EEG electrodes may be shaped, sized, or constructed to enhance collection of surface electrical activity and/or to facilitate collection of one or more of the frequency bands described herein, such as frequency bands associated with tonic phase and or clonic phase muscle activation.

In some embodiments, electrical signals resulting from muscle-related activity, including those collected from one or more head or scalp electrodes, are processed and used to determine characteristics of seizures, such as the dynamics of one or more seizure or seizure-related events. Seizure dynamics and/or other characteristics of seizure events measured from muscle-related muscle activity may then be included in a quantitative summary of detected activity. That information may then be provided to one or more caregivers. For example, a statistical summary of characteristics of detected seizure or seizure-related activity may be created and may include, by way of nonlimiting example, the duration of phases or parts of a seizure, including the tonic phase, clonic phase, entire seizure, and any combinations thereof. In some embodiments, the intensity or normalized intensity of signals derived from one or more phases of a seizure or of an entire seizure is also determined. In some embodiments, other characteristics associated with seizure or seizure-related events, including, for example, statistical metrics of clonic-phase bursts, such as described in Applicant's copending U.S. application Ser. No. 14/920,665, filed Oct. 22, 2015 and titled “Method and Apparatus for Detecting and Classifying Seizures,” are also determined. For example, methods explicitly described herein are sometimes executed together with routines described in the aforementioned reference and/or other references incorporated herein, including, for example, routines suitable to count clonic-phase bursts, which may also be referred to herein as clonic-discharge bursts. For example, one or more of the analysis routines herein may define endpoints in time over which clonic-phase bursts may be counted and analyzed.

In some embodiments, a statistical summary of characteristics of seizures or seizure-related events is provided to a caregiver, such as may be used to diagnose a condition of a patient. Or, such information may be used by a medical professional to select whether a patient may be further tested in order to diagnose the patient as suffering from a medical condition, such as epilepsy or another condition, such as PNES. In some embodiments, information derived from analysis of muscle-related electrical signals is further provided to a caregiver together with other information derived from signals directly manifested from the brain. In some embodiments, such information is provided to a caregiver for post-processing review of detected seizure activity. However, in other embodiments, such information may be provided to one or more caregivers in real-time while monitoring a patient.

In some embodiments, systems and methods described herein may be used to monitor a patient, such as to detect seizures, including, for example, GTC seizures as well as other seizures, including some that may result from conditions other than epilepsy. For example, some seizures that may share one or more characteristics with GTC seizures commonly associated with epilepsy, such as increased muscle activity or increased repetitive muscle activity, can also be detected using embodiments herein. For example, in some embodiments, PNES events are detected and classified as events that may be indicative of a patient condition other than epilepsy. In some embodiments, seizure activity may be detected and classified as resulting from a complex-partial seizure. For example, in some embodiments, sensors are disposed on both sides of a patient's body, including, on or near patient peripheral muscles of the patient and/or on or near muscles of the head and/or scalp of the patient. And, systems and methods herein may determine if detected seizure activity is symmetric or asymmetric with respect to the patient's body.

In some embodiments, seizure detection and analysis systems herein are configured to analyze an electrical signal in order to determine the dynamics of a seizure. Such analysis may further be accomplished automatically without requiring a visual inspection or manual analysis of signal data. Studies have shown that the power characteristics of muscle-related electrical signals of the tonic phase and the clonic phase may be located in different frequency bands, such as a low frequency band and a high frequency band. And, in some embodiments, the power generated in the tonic phase and in the clonic phase is extracted using different frequency bands, thus allowing the dynamics of each phase to be analyzed separately or in relation to each other, such as by calculating a ratio between frequency bands. For example, a ratio between the power of the low and high frequency bands of muscle-related electrical activity provides an improved basis for detecting seizures and/or for determining seizure characteristics, such as the dynamics of the seizure. Accordingly, systems herein are capable of analyzing the dynamics of a seizure, detecting a seizure, or both.

In some embodiments, one or more channels of an existing EEG system are modified to configure the one or more channels for analysis of muscle-related electrical signals, and the one or more channels are used to detect seizure activity, such as may be used to log detection of seizures or to initiate a system alarm. Additional channels of the EEG system may be used for additional analyses. For example, some methods herein provide one or more sensor units configured for collecting muscle-related electrical signals from one or more head or scalp electrodes and for executing one or more seizure-detection routines based on the collected muscle-related electrical signals. The one or more seizure-detection routines may execute when performing an EEG analysis, such as to provide an improved mechanism for alerting a caregiver of a detected seizure. And, in some embodiments, systems herein may include one or more sensor units specifically designed for collecting muscle-related electrical signals from one or more head or scalp electrodes and configured to allow a user to move comfortably, including while executing daily tasks in a home environment. For example, sensors configured for collecting muscle-related electrical signals from one or more head or scalp electrodes and for executing one or more seizure-detection routines may be part of a mobile sensor configured to wirelessly send electrical signals to one or more base station or reference device for the purpose of monitoring a patient for seizure activity and/or for the purpose of collecting and recording muscle-related electrical activity data for further analysis.

In some embodiments, seizure detection and analysis systems include one or more processors, each of which may include one or more modules. A module, as used herein, may, for example, comprise a series of instructions stored on a computer-readable, or an electronic storage medium storing program code, or a memory unit storing instructions that is coupled to an associated dedicated processing unit for execution of the instructions. A module may be a plugin unit, stand-alone set of instructions or program code, or may be a part of or an integral part of a larger component of a processor. Different modules of a processor may be stored in separate portions of memory or in a common portion of computer memory.

In some embodiments, seizure detection and analysis systems include one or more analysis modules. For example, each of a group of analysis modules may be configured to analyze a collected signal within a certain frequency band—such as a frequency band indicative of tonic phase seizure activity or clonic phase seizure activity, as described herein. For example, in some embodiments, seizure detection and analysis systems herein include a first analysis module configured to analyze a collected signal within a first frequency band, such as a first frequency band of about 32 Hz to about 512 Hz. A second analysis module may also be provided, the second analysis module being configured to analyze a collected signal within a second frequency band of about 2 Hz to about 16 Hz.

Systems herein may further include one or more evaluation modules. In one example, an evaluation module is configured to receive a first output signal from the first analysis module, a second output signal from the second analysis module, or both a first output signal and a second output signal. For example, in some embodiments, an evaluation module is configured to receive each of a first output signal and a second output signal and calculate a ratio between the first output signal and the second output signal. That ratio may then be compared to a threshold ratio in order to detect a seizure event, identify one or more time points in seizure dynamics, and/or define an amplitude or power threshold used in additional calculations, or used for any combination of the aforementioned applications.

In some embodiments, relative amplitudes or amounts of muscle-related electrical signals collected from head or scalp electrodes are enhanced by placing one or more EEG electrodes close to the temporalis or frontalis muscle, selectively filtering a signal derived therefrom, or both. For example, in some embodiments, an analysis module receives a signal processed using one or more high-pass or other filters configured to select one or more parts of a signal wherein muscle-related signal components have significant amplitude or where muscle-related signal components have significant relative amplitude as compared to signal components directly manifested within the brain. For example, the second analysis module described above may be configured to receive a signal derived from an EEG electrode wherein one or more high pass filters or other filters have been applied to remove at least a portion of low frequency components of an electrical signal such as may be associated with the delta band or theta band of an EEG signal. For example, a high-pass filter may be characterized by a cut-off frequency ranging from about 2 Hz to about 8 Hz. In some embodiments, within that range, a cut-off frequency may be about 2 Hz, about 3.5 Hz, about 4.5 Hz, about 6 Hz, or about 8 Hz.

According to some embodiments of seizure detection and analysis systems herein, a seizure detection module is configured to compare a ratio to a first threshold value and to generate an event signal if the ratio exceeds that threshold value, which is sometimes herein be referred to as a threshold ratio for seizure detection. According to some embodiments, a seizure detection module is configured to further compare one or more output signals of analysis modules herein (e.g., the first analysis module and second analysis described above) to another threshold value and to generate an event signal if the output signal exceeds the threshold value.

Accordingly, systems herein may be configured to detect a seizure, such as a tonic-clonic seizure, by analyzing the power ratio within the two frequency bands, by analyzing the amplitude or power of one or more output signals, or a combination of both. For example, in some embodiments, false positives are reduced by determining the power of high frequency components of a collected signal and determining if the power is above a threshold level. Advantageously, this may reduce a need for measuring multiple signals in order to detect a seizure with a low false positive detection rate. And, for example, in some embodiments, methods herein provide for seizure detection while achieving a low false positive detection based exclusively on processing of muscle-related electrical activity. For example, in some embodiments, systems herein provide one or more sensor units that may comprise or consist of electrodes configured for collecting and processing of muscle-related electrical activity.

According to some embodiments, a time-point extraction module is configured to compare one or more output signals to one or more threshold values and to determine a starting point of a phase in the detected seizure based on times when a threshold value was met or exceeded. According to some embodiments, the time-point extraction module is further configured to apply a zero-crossing function to one of the output signals and to count the number of crossings within a predetermined time window. According to some embodiments, the time-point extraction module is configured to compare the count to a threshold value and to determine at least one time point of the detected seizure based on times when the threshold value was met.

Accordingly, a duration length of a whole seizure may be determined, and different phases of the seizure may be determined, such as automatically, with improved accuracy and precision and without requiring a visual inspection of individual seizure data, thereby providing a standardized method for detecting the starting point and the end point and thereby the length (time period) of the seizure. Importantly, this analysis may be done automatically without relying on visual inspection of data by a trained epileptologist or seizure-care specialist. Such embodiments may provide a distinct advantage over existing technologies for a number of reasons. For example, as described above, detailed analysis of the dynamics of a seizure may be achieved using efficient low-cost systems and methods.

In some embodiments, detection systems and analysis methods herein are applied during real-time monitoring of a patient for seizure activity. For example, in some embodiments, the length of a seizure is provided to a first care responder so that they may be able to assess a duration of a seizure. Or, other characteristics of a detected seizure are provided to a first care responder. And, for example, if a patient seizure has lasted for longer than a certain duration, a first care responder may be provided instructions that a preferred mode of care may be to hospitalize the patient (if the patient is not already in a hospital) or to monitor the patient for an extended period of time, such as a time to verify that post-seizure nervous system suppression has not put the patient at adverse risk of further effects from a seizure. For example, in some embodiments, one or more signals instructing a caregiver of a course action (e.g., instructions based on automatic analysis of seizure semiology) are routed through a systems base station or reference device in communication with a mobile sensor unit worn by a patient. Of course, in some such scenarios, it is critically important that such information is provided automatically as first care responders will likely not be trained to perform seizure semiology nor would they have the time or resources to do so in any practical manner. Systems herein that measure semiology automatically, including some wherein, for example, a detection system may only record/log data after a seizure has been detected or in cases where tonic phase detection may be obscured (as further described below), may be particularly suited for such purposes.

In some embodiments, by counting a number of threshold crossings, such as a number of crossings with a hysteresis, in the high frequency band, the starting point and the end point of the seizure is determined in a precise and effective manner. The end point of the tonic phase and thus the starting point of the clonic phase may likewise be determined in a precise and effective manner by using the low frequency band. By using one of the frequency bands with either the other frequency band or the ratio, the time points may be determined in a more accurate manner. In some other embodiments, a number of crossings is counted, preferably the number of the crossings with a hysteresis, in the high frequency band which could be used to determine one or more of the time points.

According to some embodiments of the invention, an analysis module is configured to calculate an average signal within a predetermined time window, and wherein an evaluation module is configured to calculate a slope of the average signal during the beginning of the seizure.

Studies have shown that the initialization of a tonic-clonic seizure begins with a gradual increase in power during the tonic phase which may be described as a slope. In some embodiments, a slope parameter individually and/or along with other calculated parameters might be used to distinguish between an epileptic seizure (e.g., a GTC seizure) and a PNES (psychogenic non-epileptic seizure) or used to predict other characteristics of a detected seizure. For example, in some embodiments, if a slope for a change in power over time during the tonic phase exceeds a threshold slope, associated data will be flagged or marked as possibly associated with a PNES event. In some embodiments, a threshold slope for a change in power over time during the tonic phase is combined with other data associated with clonic discharge bursts. For example, changes in the slope of power over time during the tonic phase together with one or more characteristic patterns of PNES, such as a pattern of timing of clonic discharge busts and how such clonic discharge bursts change during later stages of the clonic phase of a seizure, may be used to provide a powerful approach for PNES detection. Notably, such capability is distinctly lacking in existing systems and provides a distinct technological advantage over other EEG systems or other prior art systems for seizure detection.

According to some embodiments, an analysis module is configured to apply a Fourier transformation function to the collected signal, and where an evaluation module is configured to calculate the median frequency for a predetermined time window based on the transformed signal. These median frequencies are used to generate a power density spectrogram for the collected signal or signal derived therefrom.

According to some embodiments of the invention, an analysis module is configured to determine the coherence between two simultaneous oscillatory activities in a first collected signal and a second collected signal. The coherence may, for example, be used to describe the dynamics of the energy in collected signals in the clonic phase as well as the energy of each clonic-discharge burst. For example, by using a timer, counter, and a peak detector, the amount of energy in the bursts and the number of clonic discharge bursts may be determined, as well as the number of silent periods between the discharge bursts.

According to one embodiment of the invention, a processor unit is configured to receive a control signal from a detection unit configured to detect a seizure, and where the processor unit is configured to record the sensed signal for a predetermined time period at least after receiving the control signal. According to a specific embodiment of the invention, the time-point extraction module is configured to determine at least the length of the seizure based on the collected signal.

In some embodiments, systems record/log data for about four minutes (two minutes prior and two minutes after a seizure is detected) or for some other suitable time period. For example, in some embodiments, the system only records/logs data for about two minutes after the seizure is detected or for some other suitable time period. This reduces the amount of data that needs to be recorded and analyzed, thus reducing the energy consumption in the processor unit. By only logging/recording a limited amount of data, the system may be more readily implemented in a small portable device which is powered by batteries and is capable of being fixed or disposed in relation to the body of a user.

Studies have shown that for many seizure patients, there is an inverse relationship between the length of one part of the tonic phase and the length of the clonic phase which may be described by using a linear or non-linear function. This relationship may be used to estimate the length of the entire seizure, in particular in systems which only records/logs data after a seizure has been detected or in cases where tonic phase detection may be obscured for one or more reasons, such as in cases where extensive non-seizure movement at times near seizure onset makes precise estimate of the starting point of a seizure difficult. In such cases, for example, the length of the tonic phase is estimated based on the length of the clonic phase. Accordingly, in some embodiments, an entire duration length of a seizure is estimated entirely from data recorded during the clonic phase of a seizure, even when the entire seizure period has not been recorded or where this data may be obscured for other reasons. Moreover, this estimate may be made available in a timely manner, including during the course of treatment for a seizure event. And, as described previously, such embodiments may be particularly useful for providing information about seizure dynamics to caregivers, including, in some cases, first responders responding to a reported seizure or seizure-related event.

In some embodiments, methods herein include analyzing the dynamics of a seizure, such as a tonic-clonic seizure, characterized in that: a recorded signal is filtered and analyzed within a first frequency band and a second frequency band, and the two output signals are evaluated by calculating a ratio between the first output signal and the second output signal. For example, a first frequency band ranges from about 32 Hz to about 512 Hz, and a second frequency band ranges from about 2 Hz to about 16 Hz.

This provides a more accurate and reproducible method for determining the characteristics and thus the dynamics of the seizure without requiring a visual inspection of individual seizure events, since studies have shown that the power characteristics of the tonic phase and the clonic phase are typically mainly located in two different frequency bands: a low frequency band and a high frequency band. This method allows the power generated in the tonic phase and in the clonic phase to be extracted using different frequency bands, thus allowing the dynamics of each phase to be analyzed separately or in relation to each other. The power in the different frequency bands may also be used to detect a seizure, e.g., the onset of a seizure. This enables the method to be implemented as a seizure detection algorithm and/or a seizure analyzing algorithm suitable for a small battery powered portable device located on or near a user.

According to some embodiments, a ratio between the power generated in high and low frequency bands is compared to a threshold value for the ratio, and an event signal may be generated if the ratio exceeds the threshold value for the ratio. Such embodiments are used as a seizure detection method capable of detecting a seizure, particularly the onset of a seizure, by analyzing the ratio between the power generated in the high and low frequency bands. The number of false positives may be further reduced by ensuring that the value of another parameter also exceeds another threshold value. This parameter may be an average value of the power in the high frequency band or in the entire frequency range of the sensed signal. This configuration facilitates methods that may not only be used to detect seizures, but that may also be used to analyze the dynamics of a detected seizure.

According to some embodiments, at least one of a starting point and an end point is determined based on the two output signals. For example, one or more of the output signals may be compared to a threshold value for determining the starting point or the end point of seizures or phases of a seizure. This facilitates methods to determine the length of the whole seizure as well as the length of the different phases in a seizure in a standardized manner, thereby eliminating the need for a visual analysis of the signal. These time points are then used to determine the dynamics of the seizure in combination with other characteristics calculated or extracted from the sensed signal. For example, in some embodiments, trends in one or more parameters or characteristics of data over time throughout a seizure or individual parts of a seizure are determined. And, such parameters can be used to identify changes in the seizure that may be indicative of normal and/or abnormal recovery from a seizure.

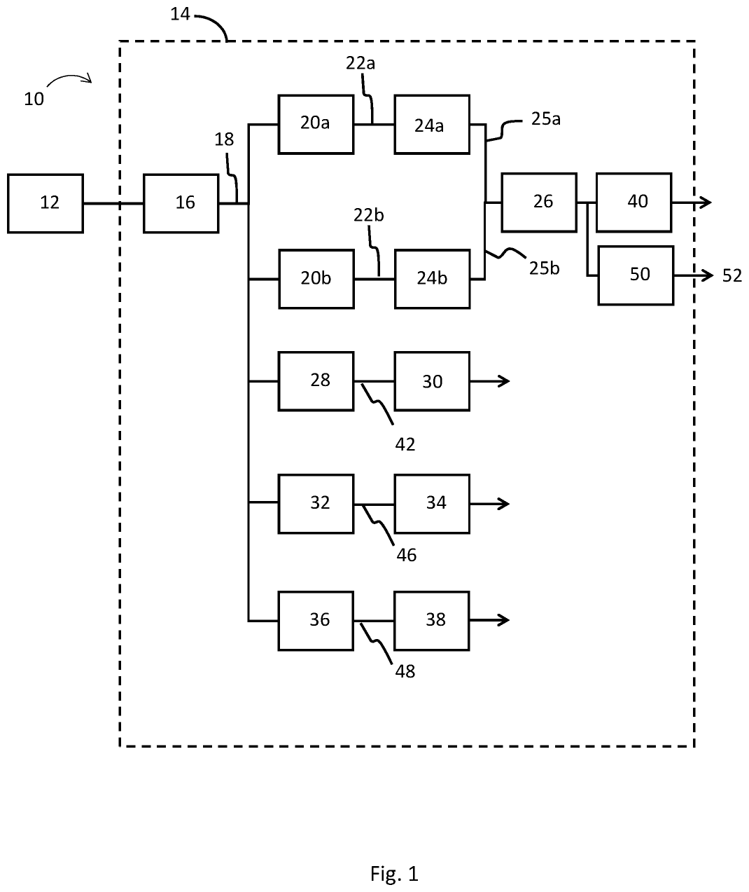

FIG. 1 illustrates embodiments of a system 10 configured for collection of electrical signals originating from a person. The system 10 comprises one or more electrodes 12 disposed on or near the body of a person, e.g., a person suffering from seizures caused by various abnormal neurological activities, such as, but not limited to epilepsy. In some embodiments, the one or more electrodes 12 may be part of a sensor unit placed on or near one or more peripheral muscles of a person, such as the biceps, triceps, hamstrings, quadriceps, or other suitable muscles of a person and any combinations thereof. The sensor unit may be configured to measure an electrical signal from the body of a person. The one or more electrodes 12 may, for example, comprise a number of surface electrodes arranged at strategic positions on the surface of one or more muscles or arranged in or near an outer surface of a sensor housing (not shown) which is configured to lie against the surface of a muscle and to be mounted or fixed to the muscle. In some embodiments, the one or more electrodes 12 may comprise a group of electrodes, the group of electrodes including each of a common electrode and a pair of detection electrodes. A group of electrodes may further be configured for executing bipolar collection of an electrical signal, such as may be used to improve discrimination between signals originating from one or more patient's muscles and noise or contaminant electrical signals.

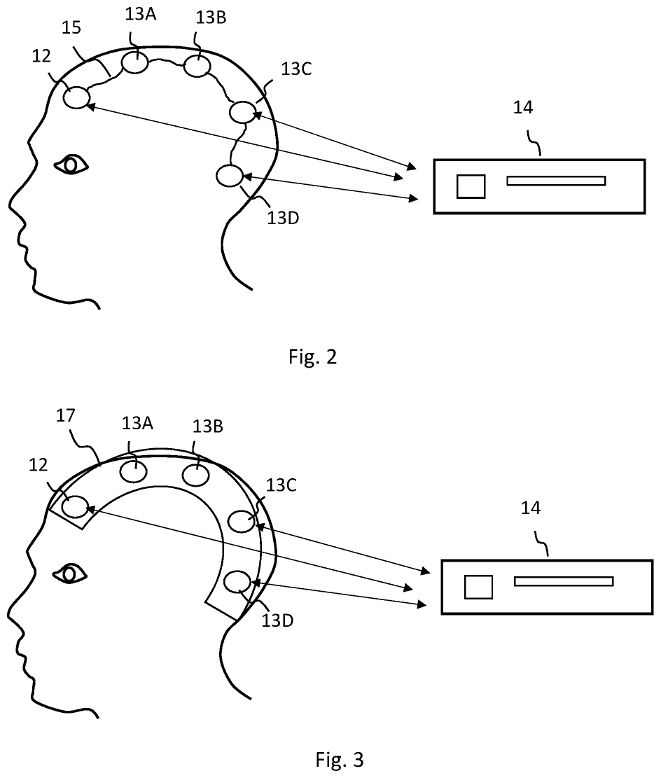

In some embodiments, as shown in FIGS. 2-5, the one or more electrodes 12 are EEG electrodes configured to collect an electrical signal generated from the head or scalp of a patient. For example, the one or more electrodes 12 may comprise one or more surface electrodes configured for placement at defined positions along the head or scalp of a patient, such as one or more of the F7, F8, T3, and T4 positions.

The one or more electrodes 12 may, for example, comprise Ag/AgCl electrodes, such as reusable sintered or disposable electrodes, or other suitable electrodes may be used. In some embodiments, the one or more electrodes 12 may comprise one or more electrodes woven or configured for use as part of a helmet or headset and may include any suitable number of electrodes. For example, the one or more electrodes 12 may be part of a headset designed for high spatial resolution imaging of brain activity and which may include a large number of electrodes or may include a smaller number of electrodes, such as may be configured for use in some ambulatory systems for seizure detection and analysis. In some embodiments, the one or more electrodes 12 are part of a sensor unit further comprising processing components, e.g., filters, amplifiers and bias circuits for conditioning a collected signal before providing the sensed signal to other components of the system 10. For example, the one or more electrodes 12 or a sensor unit including the one or more electrodes 12 may be configured to send collected signals to a processor unit 14, which may receive a collected signal via a wired or a wireless connection and process the received a received signal as further described herein. Alternatively, a sensor unit may include a processor configured to perform one or more processing operations, such as filtering to provide one or more signals in a selected frequency band, analysis of signals in a selected frequency band to provide one or more output signals, evaluation of output signals, or other operations herein. And, fully or semi-processed data may then be provided to one or more other processors for additional analysis. For example, in one embodiment, a sensor unit may perform various operations suitable to detect a seizure or seizure event. However, semiology of a detected seizure may be performed using one or more additional processors, such as may be included in one or more base stations or caregiver devices.

In some embodiments, the processor 14 is configured to receive and process at least part of signals collected from a plurality of electrodes, such as a sampled or filtered part of a collected signal. For example, as shown in FIG. 2, which shows an embodiment of system 10, one or more electrodes 12 may be positioned on the head or scalp of a patient, such as over the frontalis muscle located on the patient's forehead. A signal collected therefrom may include one or more signal components manifested from a patient's muscles. As also shown in FIG. 2, additional electrodes (13A, 13B, 13C, and 13D), which may be similarly or differently configured from the one or more electrodes 12, may likewise be positioned on the head or scalp of the patient, such as at a distance from the frontalis or temporalis muscles of a patient. The processor 14 may receive signals from each of the plurality of electrodes (12, 13A, 13B, 13C, and 13D).

In the embodiment shown in FIG. 2, the electrodes 12, 13A, 13B, 13C, and 13D may be connected together, such as with wiring 15, which may be used to hold the electrodes together and/or to route electrical signals between electrodes or groups of electrodes. In some embodiments, as shown in FIG. 3, the electrodes 12, 13A, 13B, 13C, and 13D may be part of a frame 17, which may be adjustable or fixed in shape and/or orientation. In some embodiments, as shown in FIG. 2 and FIG. 3, the electrodes 12, 13A, 13B, 13C, and 13D may wirelessly communicate signals with the processor 14. However, in other embodiments, a wired connection may sometimes be used. Signals from the electrodes 12, 13A, 13B, 13C, and 13D may be sent from one common transceiver (not shown), such as may be part of a frame. Alternatively, individual electrodes among the one or more of the electrodes 12, 13A, 13B, 13C, and 13D may include an individual transceiver and independently send signals to the processor 14.

In some embodiments, the one or more electrodes 12 are included among a set of commercially available electrodes commonly used for EEG recordings. For example, some systems herein that analyze collected signals for signatures of muscle-related electrical activity advantageously use existing EEG equipment that has been modified for performing an analysis of muscle-related signals. Such modification may comprise addition of one or more hardware components, software components, or both. For example, in some embodiments, the processor 14 is configured to analyze EEG signals directly related to brain activity, and further configured to analyze one or more muscle-related electrical signals received using a client software application downloaded thereon. An application may, for example, be provided as hardware, software, firmware, or any combinations thereof.

In some embodiments, systems herein comprise a processor configured to analyze electrical signals provided from head and scalp electrodes, wherein the processor is configured to execute each of one or more routines for analyzing electrical signals manifested directly in the brain and also to execute one or more other routines for analyzing electrical signals related to seizure activity based on muscle-related electrical signals. In some embodiments, systems may comprise a tangible computer medium including instructions for executing one or more routines for analyzing electrical signals related to seizure activity based on muscle-related electrical signals.

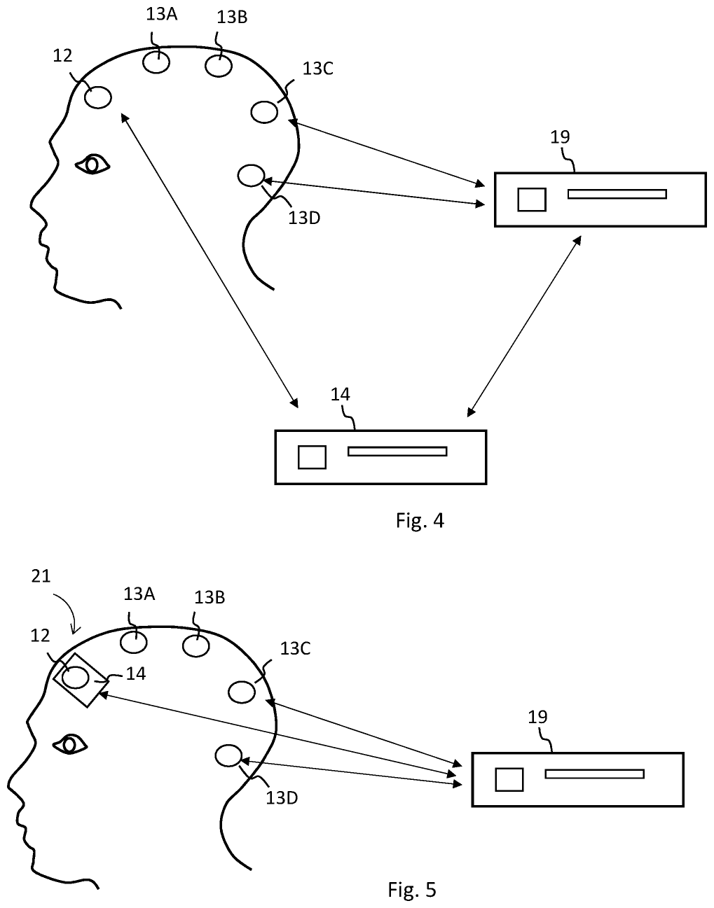

In some embodiments, the processor unit 14 is physically separated from other machinery, such as conventional EEG machinery, that may or may not be part of systems herein. For example, as shown in FIG. 4, the processor unit 14 is configured to receive a muscle-related electrical signal from the one or more electrodes 12. Other signals (e.g., as may be collected from electrodes 13C and13D) may be sent to a physically or logically separate processor unit 19. The various processors 14, 19 may be in wired or wireless communication with each other or with separate units for respective signal display if such capability is not included in one or more of the processors 14, 19.

In some embodiments, the one or more electrodes 12 are part of a sensor unit 21 (as shown in FIG. 5), the sensor unit 21 further comprising the processor 14 or one or more of the modules described therein. For example, in some embodiments, one or more of the modules 20, 24, 26, 28, 30, 32, 34, 36, 38, 40, and 50 may be part of the sensor unit 21. Sensor unit 21 may include one or more transceivers (not shown) configured to wirelessly provide sensed electrical signals from a patient muscle, processed data determined therefrom, or both to the processor unit 19. Processor unit 19 may, for example, be configured to receive a processed signal from sensor unit 21 and EEG-signals from one or more of the additional electrodes (13A, 13B, 13C, and 13D). Alternatively, the processor unit 19 may comprise a base station or remote caregiver device, designed to receive a signal from the sensor unit 21. And, in some embodiments, sensor unit 21 may operate independently of the electrodes 13A, 13B, 13C, and 13D, or other electrodes configured for collecting a signal manifested from brain activity.

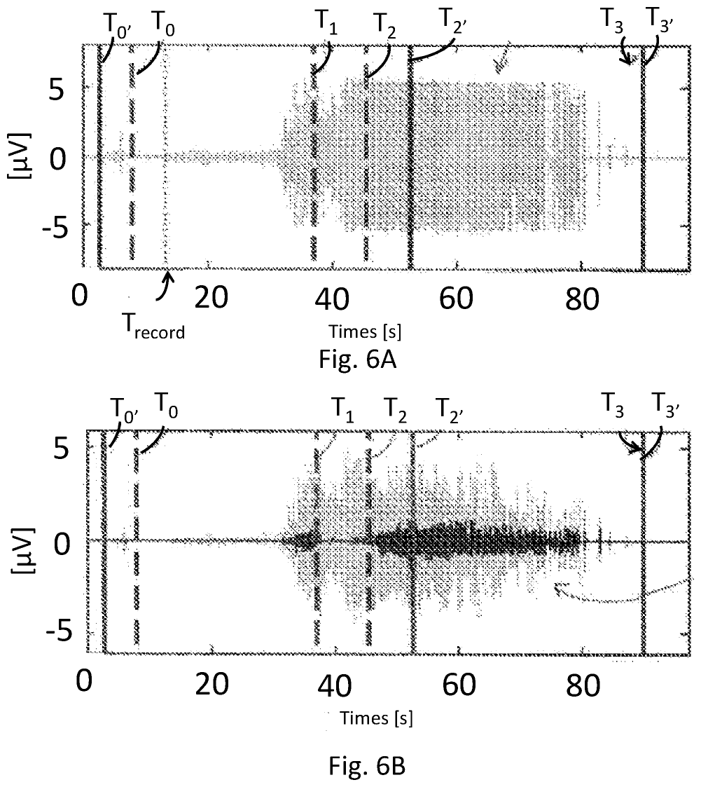

In some embodiments, the processor unit 14 is configured to receive muscle-related electrical signals collected or sensed from the electrodes 12 and to analyze the collected muscle-related electrical signals to determine a number of characteristics characterizing the dynamics of a seizure. The processor unit 14 may further include one or more units of memory and may be configured to store at least a portion of the collected signal. For example, as shown in FIG. 1, the processor unit 14 comprises a sampling module 16 configured to sample, e.g., oversample, the sensed signal at a predetermined or adjustable sampling frequency in order to provide a sampled signal 18 (shown in FIG. 6A, for example). The sampling module 16 includes one or more filters for filtering out unwanted frequencies and/or biasing the sensed signal, e.g., an anti-aliasing filter may be included. In some embodiments, the sensed signal is filtered using an anti-aliasing filter having a frequency band of about 2 Hz to about 512 Hz. In some embodiments, one or more low pass filters, high pass filters, or both may be used to remove unwanted frequencies and limit frequencies of a collected or sensed signal. Filtering may be accomplished by software or electronic circuit components, such as bandpass filters (e.g., Baxter-King bandpass filters) suitably weighted. However, such description should not be interpreted as limiting methods herein to filtering using either software or electronic circuit components. For example, in some embodiments, analog or digital signal processing techniques and/or combinations of analog and digital signal processing is used. In some embodiments, including some embodiments wherein the one or more electrodes 12 are head and/or scalp electrodes, the sensed signal is filtered with a lower frequency cut-off of about 2 Hz, about 3.5 Hz, about 4.5 Hz, about 6 Hz, or about 8 Hz.

In some embodiments, the processor unit 14 is configured to process a sensed signal within a plurality of time windows. For example, a sensed signal is broken up into a number of time windows T_{w} having a predetermined width N. The time windows T_{w} may be overlapped with an overlap M, such as a predetermined overlap. In a preferred embodiment, the sampled signal is processed using at least one time window T_{w} of 0.2-4 seconds which overlaps the next time window with an overlap M of about 10% to about 90%.

In some embodiments, sampled signal 18 (shown in FIG. 6A), is then transmitted to one or more filter modules 20a, 20b configured to filter out the sensed signal within different frequency bands. The bandwidth of each frequency band is determined according to one or more criteria defining a characterizing pattern for one or more of the phases in the seizure and/or to help discriminate muscle-related electrical signals from electrical signals originated directly from the brain. In some embodiments, a first filter module 20a is configured to filter out the sampled signal within a high frequency band B_{HF} which may comprise the majority of the characteristics in power (amplitudes) for a tonic-clonic seizure (an epileptic seizure) compared to the signal not containing a seizure. A second filter module 20b is configured to filter out the sampled signal within a low frequency band B_{LF} thereby focusing remaining portions of this band on frequencies suitable for characterizing power (amplitudes) of the clonic phase. For example, in some embodiments, the high frequency band B_{HF} has a frequency band of 32-512 Hz, preferably 64-256 Hz, and the low frequency band B_{LF} has a frequency band of 2-16 Hz, preferably 2-8 Hz. FIG. 6B shows the filtered signal 22 in the high frequency band B_{HF} (black) and in the low frequency band B_{LF} (grey).

In some embodiments, one or more of the aforementioned bands, such as the low frequency band B_{LF}, is additionally selected to increase discrimination of muscle-related activity from brain-related electrical activity. In some embodiments, the high frequency band B_{HF} includes frequencies ranging from about 32-512 Hz, preferably about 64-256 Hz, and the low frequency band B_{LF} includes frequencies ranging from about 2-16 Hz, about 3.5-16 Hz, or about 4.5-16 Hz. Generally, in embodiments where signal may be collected from electrodes positioned on the head and/or scalp of a patient, depending on relative levels of brain activity and the particular position of associated electrodes on the head or scalp of a patient, a low frequency band can exclude a lower boundary portion of signal as compared to lower frequency bands used in related algorithms, such as related algorithms used for collected signals from other muscles of the body, such as peripheral muscle of a patient (e.g., the biceps, triceps, deltoid, quadriceps or other suitable peripheral muscle). For example, in some embodiments, to increase sensitivity for detection of muscle-related electrical activity, it may useful to exclude as much as about 75% to about 100% of the delta band from the sampled signal 18. In some embodiments, it may useful to exclude as much as about 25% to about 50% of the theta band from the sampled signal 18.

The filtered signal 22a, 22b may then be transmitted to one or more analysis modules 24a, 24b configured to calculate one or more parameters of a seizure. In some embodiments, the one or more parameters are used to determine the dynamics of a seizure. The one or more analysis modules 24a, 24b are configured to analyze the power (amplitude) of the sensed signal within each frequency band B_{LF}, B_{HF}. In some embodiments, one or more of the analysis modules 24a, 24b use a wavelet, a Fourier, a chirplet or another suitable transformation function to determine the power content within each frequency band. The one or more analysis modules 24a, 24b may comprise a rectifying sub-module (not shown) connected to a smoothing sub-module (not shown) configured to generate a transformed output signal 25a, 25b, as shown in FIG. 7. In some embodiments, one or more of the filtered signals 22a, 22b are processed using a time window T_{w} of 2 seconds, or some other suitable window may be used. In other embodiments, one or more of the filtered signals 22a, 22b are processed using a time window T_{w} of 0.5 sec. Each time window may overlap the next time window by about 50%, or by some other suitable amount.

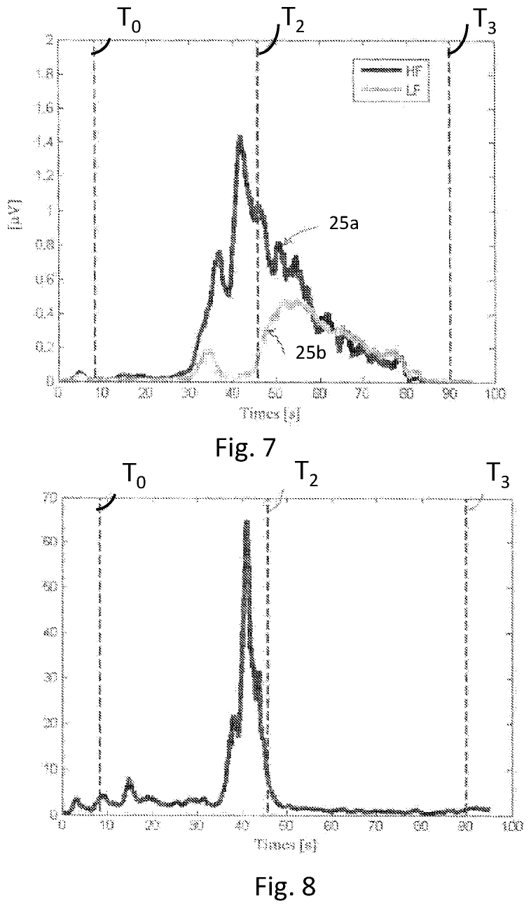

In some embodiments, the two signals 25a, 25b are transmitted to an evaluation module 26 configured to calculate the ratio HF/LF between the signals for the two frequency bands B_{LF}, B_{HF}, as shown in FIG. 8. The evaluation module 26 is configured to use any suitable method or functions to compare the value of the high frequency band B_{HF} to the value of the low frequency band B_{LF}.

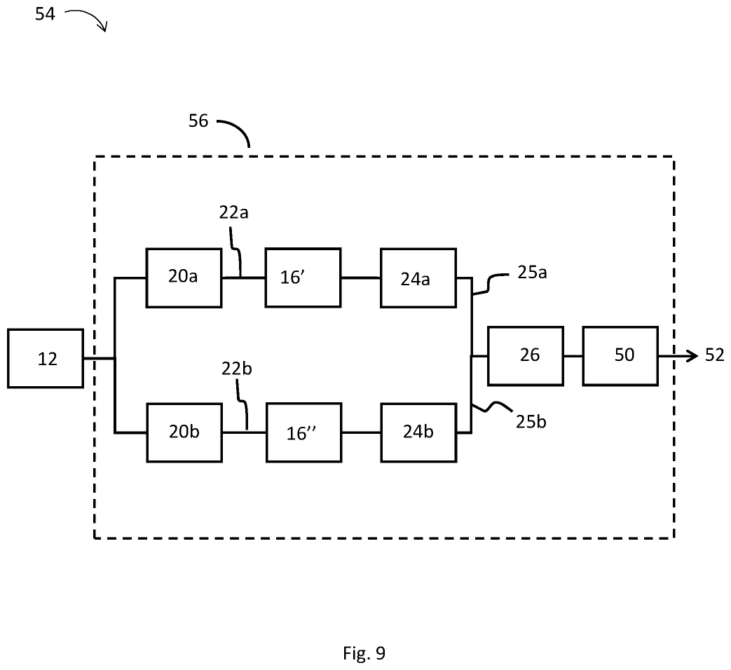

In some embodiments, a collected or sensed signal from the electrodes 12 is filtered into different frequency bands before being sampled by sampling module 16, similar to the embodiment shown in FIG. 9. In this embodiment, the filter modules 20a, 20b are connected directly to a sensor unit including the electrodes 12 and are configured to filter the sensed signal into the different frequency bands, such as bands B_{LF} and B_{HF}. The filtered signals 22a, 22b are then each transmitted to a sampling module 16′, 16″ configured to sample, e.g., oversample, the filtered signals 22a, 22b. The sampled signals are then be transmitted to the analysis modules 24a, 24b which may, in some embodiments, comprise a rectification sub-module and a smoothing sub-module. The signals 25a, 25b may then transmitted to the evaluation module 26. In this embodiment, the sensed signal may also be transmitted to the sampling modules 16′, 16″ so that the entire range of frequencies may be analyzed using one or more of the additional modules 28, 30, 32, 34, 36, 38 or other modules described herein.

In some embodiments, a signal indicating the ratio HF/LF, shown in FIG. 8, is transmitted to a time-point extraction module 40 configured to determine one or more time points of a seizure. For example, the time-point extraction module 40 may be configured to determine one or more of the starting point T₀ of a seizure, ending point T₃ of a seizure, staring point T₂ of the clonic phase of a seizure, and any combinations thereof. In some embodiments, the transformed signals 25a, 25b are transmitted to the extraction module 40 instead of or in addition to the ratio HF/LF. In some embodiments, the extraction module 40 are configured to compare the transformed signal 25b of the low frequency band B_{LF} to a threshold value for determining the starting point T2 of the clonic phase. In some embodiments, this time point is also used to define the end of the tonic phase. The starting point T2 of the clonic phase is also determined as the time when the transformed signal 25b meets a threshold value subject to one or more further conditions. For example, the starting point T2 of the clonic phase is determined as the time when the transformed signal 25b meets a threshold value subject to the additional condition that the threshold value is reached after the time point of a peak value of the ratio (HF/LF). Alternatively, the starting point T2 of the clonic phase is determined as the time when the transformed signal 25b meets an automatically calibrated threshold value wherein the threshold value is calibrated based on a power or strength of the signal P_{record} at the time of seizure detection T_{record}.

For example, in some embodiments, the values P_{record}, T_{record} are recorded based a comparison of the ratio HF/LF to a threshold ratio for seizure detection as further described in relation to the seizure detection module 50. A recorded value may be used to calibrate or select the above threshold value (i.e., the threshold value for comparing the transformed signal 25b of the low frequency band B_{LF} as may be used to determine the starting point T₂) since the time point of the maximum value of the ratio HF/LF and timing of seizure detection occur before the time point marking the tonic to clonic transition. Thus, seizure detection and the associated value P_{record} determined at the time of seizure detection T_{record} is determined and made available for use prior to its application in determining the dynamics of the seizure. Alternatively, a recorded power or signal intensity used for calibrating or determining the above threshold may be based on one or more measured powers or signal intensities for other points at or near a time when a seizure was detected, such as at about the time when the maximum value of the ratio HF/LF is achieved. For example, a threshold value may be selected based on a power that is achieved at a time when the value of the ratio HF/LF has decreased by about 25% to about 75% from its maximum value. Accordingly, in some embodiments, one or more thresholds applied in determining the dynamics of the seizure may be determined by automatic calibration based on characteristics of a detected seizure, such as the overall strength of a seizure and timing of signals in different frequency bands, thereby further improving the precision and accuracy for analysis of seizure dynamics over existing methods.

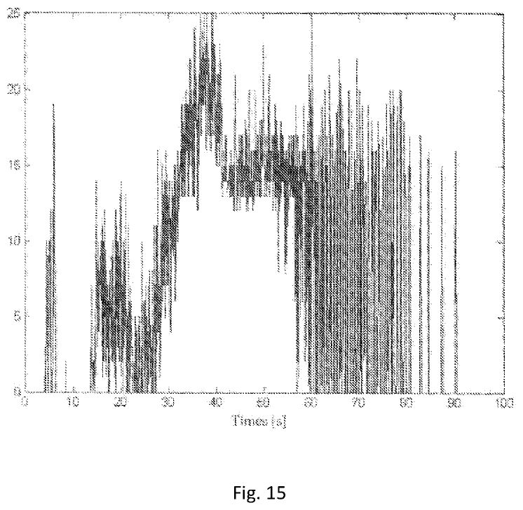

In some embodiments, the extraction module 40 may further be configured to count the number of zero-crossings of the transformed signal 25a of the high frequency band B_{HF} in a predetermined interval (time window), as shown in FIG. 15. In a preferred embodiment, the time window is 20-1024 samples (or about 0.02-1 sec.), preferably 100 samples (or about 0.1 sec.). Each time window may overlap the next time window by about 50-90%, preferably 75%. The extraction module 40 may be configured to count the number of crossings with a predetermined hysteresis threshold, e.g., of +/−25-75 μV, preferably +/−μ50 V. The extraction module 40 may then compare the counts with a count threshold value for determining the end point T₃ of the clonic phase. In some embodiments, this time point may also define the end of the seizure. The end point T₃ of the clonic phase is, in an embodiment, defined as the end of the last clonic phase burst having a count of 10 or more. The last clonic phase burst normally occurs within about 90 sec. after the seizure has been detected.

In some embodiments, the starting point T₀ of a seizure is determined by the extraction module 40 by comparing the counts (shown in FIG. 15) to a second count threshold value, which may the same or different than the count threshold value used to determine the end point T₃. For example, the starting point T₀ of the tonic phase may be defined as the time of the first burst having a count meeting a count threshold of 10 or more. For example, the first burst to occur within the seizure may occur about 30 sec or less before the seizure is detected. In some embodiments, the starting point T₀ of a seizure may be determined by the extraction module 40 by comparing the transformed output signal 25a to a threshold.

In some embodiments, the processor unit 14 is configured to limit an amount of recorded data. For example, in some embodiments, the processor unit 14 is configured to record an amount of data from the electrodes 12 of about two minutes before and after the detection T_{record} of a seizure. In other embodiments, the processor unit 14 is configured to only record data from the electrodes 12 for about two minutes after the detection T_{record} of a seizure, as described later. The seizure detection may be performed by the processor unit 14 via a seizure detection algorithm implemented in the processor unit 14 or in a second processor unit connected to the processor unit 14.

The extraction module 40 may be configured to calculate the length (time period) of the seizure and the different phases respectively based on the determined time points T₀, T2 and T₃. The processor unit 14 may be connected to a central server/unit or external device (not shown) via a wired or wireless connection and may transmit the time points T₀, T₂, T₃ and the lengths to the central server/unit or the external device for further analysis. For comparison, corresponding time points T_{0′}, T_{2′}, and T_{3′} determined by visual inspection are marked with solid lines in FIGS. 6A and 6B.

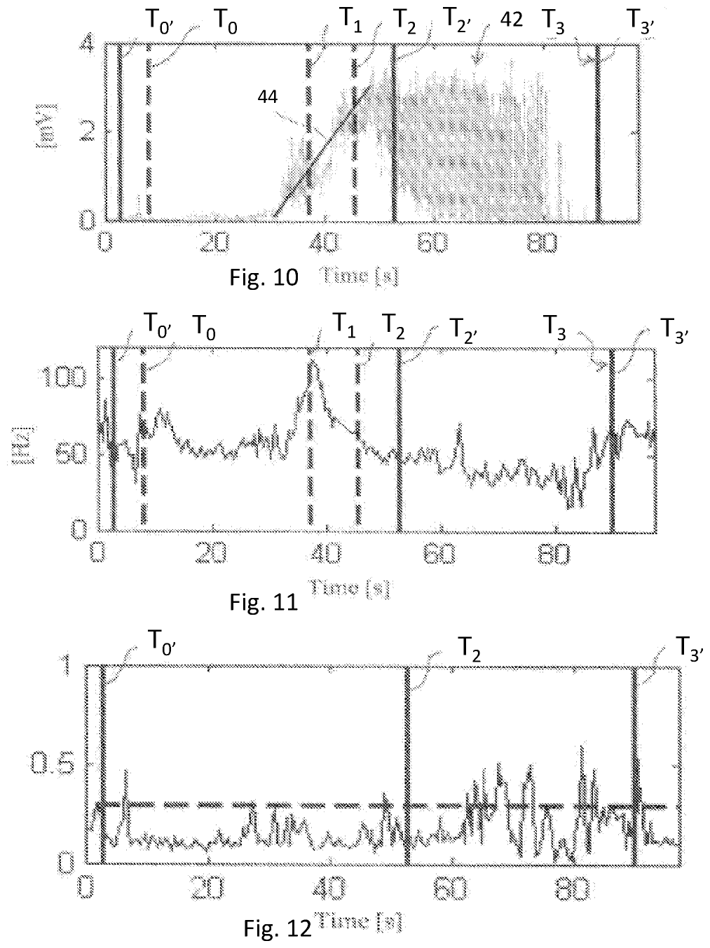

The processor unit 14 may comprise an analysis module 28 configured to calculate an averaged signal 42, as shown in FIG. 10, for the sensed signal within a predetermined time window. The second analysis module 28 may, for example, be configured to apply a root mean square (RMS) function to the sampled signal 18. The analysis module 28 may alternatively comprise a rectifying sub-module connected to a smoothing sub-module configured to generate the averaged signal 42. In a preferred embodiment, the sensed signal is processed using a time window of 1 sec. Each time window overlaps the next time window by 50%. Alternatively, this module 28 may be omitted and the sampled signal 18 may be transmitted directly to an evaluation module 30.

The averaged signal 42 may then be transmitted to the evaluation module 30, wherein the evaluation module 30 may be configured to calculate the slope 44 of the averaged signal during the beginning of the seizure (the tonic phase). The slope 44 is shown in FIG. 10. The evaluation module 30 may determine a time point T_{begin} and a corresponding value for the signal 42 at the beginning of the seizure and a time point Tₚₑₐₖ and a corresponding value for the signal 42 at the peak of the transmitted signal. The slope 44 may then be determined using a linear interpolation between the two reference points. These two time points may differ from the starting T₀ and the time point T_{record}. The time point T_{begin} for the beginning may be determined as the time point where the value of the averaged signal 42 starts to increase. In one preferred embodiment, the time point Tₚₑₐₖ for the peak value is determined as the time point of the maximum value of the ratio HF/LF. The value for the slope 44 may then be stored and reported to one or more caregivers.

In some embodiments, the processor unit 14 comprises an analysis module 32 configured to apply a Fourier transformation function to the sensed signal within a predetermined time window (epoch). The transformed signal 46 is then transmitted to a third evaluation module 34 configured to calculate the median frequency for each time window, as shown in FIG. 11. The median frequency is defined as the frequency which divides the magnitude spectrum within each time window into two parts of equal sizes (equal areas under the curve). A smoothing sub-module (not shown) may then be applied to the signal for minimizing segmentation errors. In a preferred embodiment, the sensed signal is processed using a time window of 250 ms. Each time window overlaps the next time window by 50%. The median frequencies may then be transmitted to the central server/unit or the external device.

In some embodiments, the processor unit 14 comprises an analysis module 36 configured to determine the coherence between two simultaneous oscillatory activities (a measure for the correlation in the frequency domain). The coherence is determined based on a first recorded signal, e.g., from a right-side muscle of the head or scalp of a patient, and a second recorded signal, e.g., from a left-side muscle of the head or scalp of a patient. The analysis module 36 is configured to determine the coherence between a selected activity and an adjacent activity on the left side and on the right side respectively (see, Conradsen et al., supra, which is incorporated by reference). A mean coherence may then be calculated for a predetermined time window, as shown in FIG. 12, using an averaging sub-module (not shown). In an embodiment, the sensed signal is processed using a time window of 1 sec. Each time window overlaps the next time window with 50%.

The value for the coherence 48 is transmitted to an energy calculation module 38 configured to calculate the energy of the seizure and/or the dynamics of the energy of the seizure. The calculation module 38 may comprise timers, counters and peak detectors which are configured to detect the peak values, the durations (lengths) and the number of the bursts in the seizure, e.g., in the clonic phase. These parameters may then be stored and/or communicated to one or more caregivers.

In some embodiments, the previously described time points T₀, T₂, T₃ and lengths of the different phases in the seizure is determined based on data recorded after a seizure has been detected (marked by dotted lines in FIG. 6A). The system in this embodiment is configured to record data from the electrodes 12 for about two minutes after a seizure has been detected.

Accordingly, in this embodiment, data from the electrodes 12 may not be recorded by the processor unit 14 until after a seizure has been detected T_{record} by a seizure detection algorithm. This means that data for the entire seizure period may not be recorded, leaving parts of the tonic phase unrecorded.

Studies have shown that the length of part of the tonic phase may be constant or substantially constant during the detected GTC seizures. These studies have also indicated an inverse-proportional relationship between the length of the other part of the tonic phase and the length of the clonic phase. According to the studies, a short tonic phase was followed by a long clonic phase and vise-versa.

In some embodiments, this relationship may be described using a linear function (model) implemented in the extraction module 40, thereby allowing the length of the tonic phase to be based on the length of the clonic phase, or vise-versa. The time point T₀ or T₃ may be determined using the constant period T₁-T₂ and the relationship between time periods (lengths) T₀-T₁ and T₂-T₃. Time point T₁ defines an intermediate time point in the tonic phase used in the subsequent calculations. The extraction module 40 is then able to calculate the length of each phase and thus the length of the seizure based on the recorded data. Other non-linear functions may be used to describe the relationship between time periods T₀-T₁ and T₂-T₃. This relationship may also be used to calculate the time points To and/or T₃ even if the whole seizure period has been recorded. In some embodiments, the length of the seizure may be provided to first care providers so that they may be provided a duration of a seizure. And, for example, if a patient seizure has lasted for longer than a certain duration, a first care responder may be instructed that a preferred mode of care may be to hospitalize the patient (if the patient is not already in a hospital) or to monitor the patient for an extended period of time.

In some embodiments, a seizure detection algorithm may be configured to detect a seizure, e.g., the onset of a seizure, based on the calculated ratio HF/LF. In one embodiment, the seizure detection algorithm is implemented in the processor unit 14 where the ratio HF/LF calculated by the evaluation module 26 is transmitted to a seizure detection module 50, as shown in FIG. 1. The seizure detection module 50 is configured to compare the ratio HF/LF with a threshold value of the ratio for detecting the seizure, e.g., the onset of the seizure. The seizure detection module 50 may then generate an event signal 52, e.g., an alarm signal, if the ratio HF/LF exceeds/crosses the threshold value for the ratio. In some embodiments, the threshold value may be determined based on the value P_{record}. The seizure detection module 50 may be configured to further compare at least one of the signals 25, 42, 46, 48, e.g., the transformed signal 25a of the high frequency band B_{HF} or the averaged signal 13, to a threshold before generating the event signal. The transformed signal 25a of the high frequency band B_{HF} may be averaged, e.g., rectified and smoothed, before being transmitted to the seizure detection module 50. If the ratio HF/LF and the other signal, e.g., signal 25a, exceed/are above threshold values respectively, then the event signal may be generated. The event signal may be transmitted to an alarm unit (not shown), the central server/unit or the external device. For example, in some embodiments, the event signal may activate the recording of the signal from the electrode 12, as described above.

In some embodiment, one or more of the seizure detection algorithms or other suitable algorithms described above may be implemented in a sensor unit 54. As shown in FIG. 9, the sensor unit 54 may include a processor unit 56. In some embodiments, a generated event signal 52 may activate the one or more electrodes 12 and/or processor 14, shown in FIG. 1 or trigger one or more other functions. FIG. 9 shows a simplified embodiment of the sensor unit 54 where only the filter modules 20a, 20b, the sampling modules 16′, 16″ and analysis modules 24 are shown. The sensed signal may be filtered 22a, 22b before or after being sampled, as described above. The sampling modules 16′, 16″ are configured to sample the sensed signal within the frequency bands B_{LF} and B_{HF}. The processor unit 56 may then transmit the event signal 52 generated by the sensor unit 50 to the processor unit 14. The event signal 52 then activates seizure analysis which may comprise use of one or more of the modules 16, 20, 24, 26, 28, 30, 32, 34, 36, 38, 40 or use of only modules 16, 40, 28, 30, 32, 34, 36, and 38. The processor unit 56 may further transmit one or more of the signals generated by the modules 20, 24, and 26, e.g., the ratio HF/LF and the transformed signal 25a, to the respective modules in the processor unit 14.

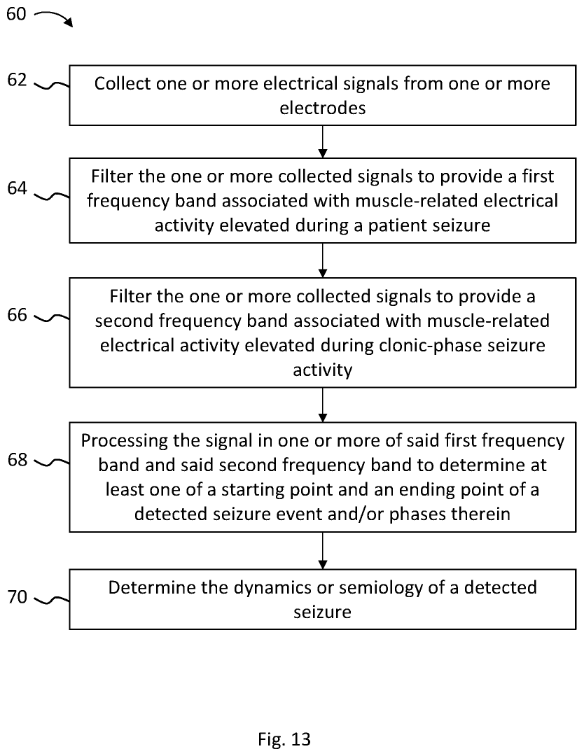

FIG. 13 illustrates an embodiment of a method 60 for determining the dynamics or semiology of a detected seizure event. In step 62, one or more electrical signals may be collected, including, for example, from one or more electrodes positioned on peripheral muscles of a patient's body, the head or scalp of a patient's body, or both. For example, in some embodiments, the electrodes 12 may be part of an EEG-headset or part of another device which may operate together with other electrodes, such as other electrodes (13A, 13B, 13C, and 13D) which may be configured to sense electrical activity derived directly from the brain. In some preferred embodiments, electrodes 12 may include one or more electrodes positioned near or adjacent one or more of the temporalis or frontalis muscles of a patient. In some embodiments, the electrodes 12 are part of a sensor unit 21, such as may include one or more transceivers, processors, or both. For example, a processor may be part of sensor unit 21 and/or a processor may be wired or wirelessly configured to receive an electrical signal collected using the senor unit 21 (e.g., a processor 14, 19 may receive an electrical signal directly or indirectly from a sensor unit 21).

As shown in step 64, a collected signal may be filtered to provide signal in a first frequency band (e.g., a first frequency band associated with muscle-related electrical activity elevated during a patient seizure). For example, the first frequency band may include one or more frequency bands that may show increased power levels during onset of a seizure and/or during the tonic phase of a seizure. In some embodiments, the first frequency band may comprise one or more frequency components ranging from about 32 Hz to about 512 Hz. Within that range, a lower frequency boundary may be about 32 Hz or about 64 Hz. Also, within that range, an upper frequency boundary may be about 256 Hz, about 128 Hz or about 64 Hz. For example, an upper frequency boundary may sometimes be selected based on a sampling rate configured for use with processor 14, which may, for example, be limited depending upon type or model of EEG-machinery.

As shown in step 66, a collected signal may be filtered to provide signal in a second frequency band, the second frequency band associated with muscle-related electrical activity elevated during clonic phase seizure activity. In some embodiments, the second frequency band may comprise one or more frequency components ranging from about 2 Hz to about 16 Hz. Within that range, a lower frequency boundary may be about 2 Hz, about 3.5 Hz, about 4.5 Hz, about 6 Hz, or about 8 Hz. Also, within that range, an upper frequency boundary may be about 8 Hz, about 12 Hz, about 14 Hz, and about 16 Hz.

As shown in step 68, the filtered signal from one or more of said first frequency band and said second frequency band may be processed to determine at least one of a starting point and an ending point of a seizure event and/or phases therein. For example, processing of the filtered signals may include analysis of the filtered signals 22a, 22b using analysis modules 24a, 24b and processing with the time-point extraction module 40. Once the time points for a detected seizure are determined, various further actions as described herein may be executed. For example, as shown in step 70, at least one of the starting and ending points of a seizure event or phases therein may be used to determine the dynamics or semiology of a seizure. That information may then be provided to one or more caregivers, such as in the form of one or more automatically generated reports.

In some embodiments, the duration length of one or more of the entire seizure duration, duration of the tonic phase of a seizure, duration of the clonic phase of a seizure or any combinations thereof are compared against corresponding duration lengths recorded historically for the patient. Alternatively, duration data may be compared against historical data for one or more patient demographic groups. For example, a patient demographic may be defined based on various characteristics, including by way of nonlimiting example, age, gender, ethnicity, weight, fitness level, or care regimen (e.g., medications provided or other treatments provided to a patient). A caregiver may then be provided with information indicating how a detected seizure and associated duration lengths may compare against typical values for a particular patient, patient demographic, or both.

In one particular embodiment, a duration length of one or more of the entire seizure duration, duration of the tonic phase of a seizure, duration of the clonic phase of a seizure or any combinations thereof may be plotted against a time that one or more drug or treatment regimen was provided to a patient, if such information has been logged. Accordingly, a caregiver may be provided information to determine whether a given drug or treatment regimen may be effective at attenuating one or more parts of a seizure. For example, a duration of the tonic phase may be plotted against a time when a treatment drug was provided or against a concentration of a drug or drug metabolite (as may be based on various pharmacokinetic properties of a given drug) if such information is available.

Still referring to step 70, in some embodiments, an amplitude or intensity of a seizure and/or individual phases of a seizure thereof may also be determined. For example, the integrated amplitude or power of the entire seizure, tonic phase of a seizure, clonic phase of a seizure, or any combinations thereof may be determined. And, in some embodiments, an amplitude or power of a seizure may be determined for one or more of the high frequency and low frequency bands described herein (e.g., output signals for each of the first and second analysis modules described herein) or for one or more high frequency and low frequency bands further described in U.S. application Ser. No. 15/491,883, filed Apr. 19, 2017 and titled, “Systems and Methods for Characterization of Seizures” the full contents of which is herein incorporated by reference. For example, in some embodiments, each of an integrated amplitude of a high frequency components of a collected signal (e.g., a collected signal ranging from about 150 Hz to about 400 Hz) and an integrated amplitude of low frequency components of a collected signal (e.g., a collected signal ranging from about 10 Hz to about 70 Hz) may be determined, wherein the temporal boundaries of integration have been established based on the seizure semiology determined herein. A ratio between the two integrated amplitude may then be compared to a ratio threshold. Studies have shown that such ratios may be used differentiate between GTC seizures and PNES events. Accordingly, in some embodiments, one or more analyses based on the semiology of a seizure may be executed, wherein the one or more analyses may be used to characterize whether a detected seizure event may be identified as either a GTC seizure or a PNES event.

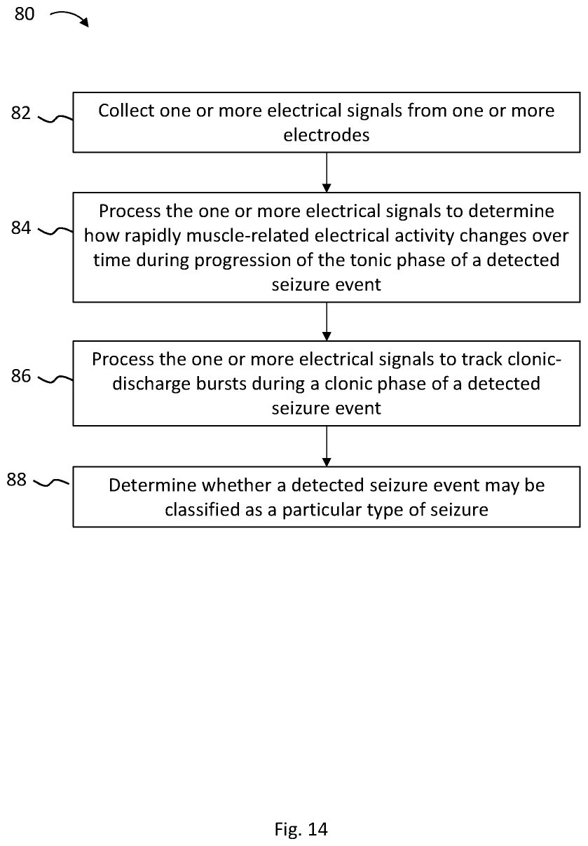

FIG. 14 illustrates some embodiments of a method 80 for classifying a detected seizure, such as being a PNES or GTC seizure. As shown in step 82, one or more electrical signals may be collected, including, for example, from one or more electrodes positioned on peripheral muscles of a patient's body, the head or scalp of a patient's body, or both. In some embodiments, any of the systems described herein in relation to FIGS. 2-5 or other appropriate systems may be used to collect the one or more electrical signals.

In step 84, one or more analysis routines may be executed to measure how rapidly muscle-related electrical activity may change over time during progression of the tonic phase of a detected seizure event. For example, as described in relation to FIG. 10, a routine for measuring how rapidly muscle-related electrical activity changes over time during progression of the tonic phase of a seizure may include steps of determining an average of a signal related to muscle-related electrical activity and calculation of a slope of the signal over time. If the slope for a change in power over time during the tonic phase exceeds a threshold slope, associated data may be flagged or marked as possibly associated with a PNES event. Thus, steps 82 and 84 may be used to identify a PNES seizure event. In some embodiments, as described below in relation to step 86, the above analysis of tonic phase signal may be combined with an additional analysis executed on clonic phase signal, that additional analysis used to further verify that a seizure event may properly be characterized as a PNES event.

In step 86, one or more analysis routines may be executed to track clonic-discharge bursts during a clonic phase of a detected seizure event. For example, one or more of any of the analysis routines described in this disclosure may be executed to identify time points for the start and end of the clonic phase of a seizure event, and to select signal to be further analyzed for detection of clonic-discharge burst activity. For example, any analysis described with respect to the time point extraction module 40 may be applied in step 86 of the method 80. In some embodiments, only the start time point of the clonic phase of a seizure may be identified. And, some portion of signal following the start of the clonic phase of a seizure, such as a predetermined portion lasting for about 1 minute to about 3 minutes, may be selected for further analysis of clonic-discharge burst activity.

Once a portion of signal is identified, one or more routines may be executed to detect clonic-discharge bursts. For example, in some embodiments, detection of clonic-discharge bursts may include rectifying a collected electrical signal and identifying peaks included within the rectified signal based on identification of regions of signal with increased signal amplitude (e.g., analyzing the signal for threshold changes in a leading and/or trailing edge of a peak). Detection may further include determining one or more of a signal to noise or amplitude of detected peaks and qualifying whether peaks are indicative of clonic-discharge burst activity based on whether individual peaks meet either or both of a threshold signal to noise or threshold amplitude and also determining if a duration width of peaks meet each of a minimum duration width threshold and a maximum duration width threshold suitable to qualify a peak as being a clonic-discharge burst. For example, in one particular embodiment, peaks that meet each of a signal to noise threshold of at least about 10 to about 20 and a duration width ranging from about 25 milliseconds to about 400 milliseconds may be qualified as clonic-discharge bursts. In some embodiments, one or more additional or alternative methods may be used to qualify a peak as being a clonic-discharge burst.

For example, in some embodiments, a collected signal may be processed to identify if one or more peaks of the signal that include an elevation in signal amplitude are detected and further determining if the one or more peaks meet one or more qualification thresholds suitable to identify that the one or more peaks are indicative of clonic-discharge burst activity. In some embodiments, the one or more qualification thresholds may include one or more duration width thresholds selected from a group of duration width thresholds including a maximum duration width threshold, a minimum duration width threshold, and a combination of both a maximum duration width threshold and a minimum duration width threshold. Qualification of peaks of signal may also include organizing a plurality of peaks into one or more groups, determining one or more aggregate property values for the one or more groups; comparing the one or more aggregate property values to one or more aggregate qualification threshold values; and determining a set of clonic-discharge bursts based on the comparing of said one or more aggregate property values to said one or more aggregate qualification threshold values. For example, included among aggregate qualification threshold values that may be used to qualify peaks in a group are one or more of a minimum deviation value calculated from duration widths of peaks, a maximum deviation value calculated from duration widths of peaks, a minimum rate of peak repetition, a maximum rate of peak repetition, a minimum regularity of one or more peak characteristics, a maximum regularity of one or more peak characteristics, and/or combinations of the aggregate qualification threshold values thereof. In some embodiments, peaks may be excluded from some groups as part of routines configured, for example, to search for particular patterns of activity. Removal of peaks and test group construction, including as related to identification of patterns in data that may be noisy or intermittent, is also described in greater detail in Applicant's U.S. patent application Ser. No. 15/539,581 filed Dec. 25, 2015 and titled, “Method and Apparatus of Monitoring a Patient for Motor Manifestations Related to Seizure Activity,” the disclosure of which is incorporated herein by reference.

Once a group of clonic-discharge bursts has been identified (e.g., as using any of the various techniques above or other suitable methods), clonic-discharge burst data may be tracked over time and used to better understand a seizure. For example, any of various statistical values of clonic-discharge burst activity may be determined and provided to one or more caregivers. In some embodiments, by way of nonlimiting example, statistical values of clonic-discharge burst activity which may be determined and provided to a caregiver may include a number of detected clonic-discharge bursts, rate of clonic-discharge bursts detection, average signal-to-noise ratio (SNR) of detected clonic-discharge bursts, spread of SNR of detected clonic-discharge bursts, average width for detected clonic-discharge bursts, spread of widths for detected clonic-discharge bursts, average duration width of periods between detected clonic-discharge bursts, spread of duration width of periods between detected clonic-discharge bursts, deviation of clonic-discharge bursts over any consecutive number of detected clonic-discharge bursts, frequency characteristics of clonic-discharge bursts, and any combinations thereof.

In some embodiments, clonic-discharge burst data may be processed to determine a linear fit for how times between detected clonic-discharge bursts changes during the course of the clonic phase of a seizure. For example, a linear fit of times between detected clonic-discharge bursts against burst number may be determined. Any of various suitable methods, such as a linear least squared regression analysis, may be used. A slope may further be characterized to one or more of minimum slope value, a maximum slope value or both to characterize a detected seizure event. For example, if a slope is found to be less than a threshold slope of about 1.0, a seizure event may be characterized as indicative of activity typically associated with PNES. Alternatively, if a slope is found to be greater than a threshold slope of about 2.0, a seizure event may be characterized as indicative of activity typically associated with a GTC seizure.

In a step 88, the results of the above analyses may be used to identify the presence of PNES seizures or GTC seizures. For example, where each of tonic and clonic phase activity is found that is indicative of PNES activity, a detected seizure event may be classified with improved confidence.

The disclosed subject matter is further described in term of the following clauses:
1. A system for determining the semiology of seizures, the system comprising:
   one or more processor units capable of receiving an electrical signal, the received electrical signal comprising at least a part of a collected electrical signal collected from one or more electrodes disposed on the head or scalp of a patient;
   a first analysis module configured to analyze at least a portion of the received electrical signal in a first frequency band within a range from about 32 Hz to about 512 Hz to provide a first output signal;
   a second analysis module configured to analyze at least a portion of the received electrical signal in a second frequency band within a range from about 2 Hz to about 16 Hz to provide a second output signal; and
   a time-point extraction module configured to compare at least one of the first output signals or said second output signals to at least one threshold value and to determine a starting point of at least one seizure event phase based on meeting the at least one threshold value.
2. The system of clause 1, at least one of said one or more processor units configured for recording at least a portion of the received electrical signal.
3. The system of clause 1, each of said first analysis module, said second analysis module and said time-point extraction module being part of a common processor unit.
4. The system of clause 1, each of said first analysis module and said second analysis module being part of a common processor unit;
   said common processor unit further configured to provide each of said first output signal and said second output signal to a second processor unit which includes said time-point-extraction module.
5. The system of clause 1 further comprising a sensor unit, the sensor unit including said one or more electrodes.
6. The system of clause 5, the sensor unit including at least one of said one or more processors, the at least one processor including each of said first analysis module, said second analysis module, and said time-point extraction module.
7. The system of clause 5, the sensor unit including at least one of said one or more processors, the at least one processor including each of said first analysis module and said second analysis module;
   the at least one processor further configured to provide each of said first output signal and said second output signal to a second processor unit which includes said time-point-extraction module.
8. The system of clause 1, said one or more processor units comprising a base station.
9. The system of clause 8, said base station configured to provide a report indicting the semiology of a detected seizure to one or more caregiver devices.
10. The system of clause 8, said base station configured to provide an automatically generated report indicting the semiology of a detected seizure to one or more caregiver devices.
11. The system of clause 1, at least one of said one or more processor units is in wireless communication with a sensor unit, the sensor unit including said one or more electrodes.
12. The system of clause 11, the at least one processor including each of said first analysis module, said second analysis module, and said time-point extraction module.
13. The system of clause 1 further comprising one or more filters for filtering said received signal in order to isolate said second frequency band, the second frequency band excluding at least a portion of the delta band, the theta band, or both.
14. The system of clause 1 further comprising one or more filters for filtering said received signal in order to isolate said second frequency band, the second frequency band including a low frequency boundary of about 3.5 Hz.
15. The system of clause 1 further comprising one or more filters for filtering said received signal in order to isolate said second frequency band, the second frequency band including a low frequency boundary of about 6 Hz.
16. The system of clause 1 further comprising an evaluation module, the evaluation module configured to calculate a ratio (HF/LF) between said first output signal and said second output signal and to generate an alarm signal if the ratio (HF/LF) meets a threshold ratio for seizure detection.
17. The system of clause 1 further comprising an evaluation module, the evaluation module configured to calculate a ratio (HF/LF) between said first output signal and said second output signal and to generate an alarm signal if the ratio (HF/LF) meets a threshold ratio for seizure detection and one or more of said first output signal and second output signals meets a threshold value.
18. The system of clause 16 or clause 17, the evaluation module being part of a processor that is physically separate from a sensor unit, the sensor unit including said one or more electrodes.
19. The system of clause 16 or clause 17, the evaluation module included in a processor of a sensor unit, the sensor unit including said one or more electrodes.
20. The system of clause 1 wherein said time-point extraction module is further configured to apply a zero-crossing function to one or more of the first output signal or the second output signal and to count the number of crossings within one or more time windows.
21. The system of clause 1 further comprising said one or more electrodes, the one or more electrodes configured for positioning at one or more of the F7, F8, T3, and T4 positions.
22. The system of clause 1 wherein said received electrical signal comprises a sampled or filtered part of said collected electrical signal.
23. The system of clause 1, said one or more electrodes included as part of an EEG headset, the EEG headset further including one or more additional electrodes configured to collect an electrical signal indicative of patient brain activity.
24. The system of clause 1, at least one of said one or more processor units configured for receiving an electrical signal from one or more additional electrodes disposed on the head or scalp of the patient, the one or more additional electrodes configured for collection of an electrical signal manifested directly from the brain.
25. A system for determining the semiology of seizures, the system comprising:
   one or more processor units capable of receiving an electrical signal collected from one or more electrodes disposed on the head or scalp of a patient, the one or more processor units configured to analyze the received electrical signal for detection of seizure events based on muscle-related electrical activity;
   a first analysis module configured to analyze at least a portion of the received electrical signal within a first frequency band to provide a first output signal, the first frequency band associated with tonic-clonic seizure activation of muscle; and
   a second analysis module configured to analyze at least a portion of the received signal within a second frequency band to provide a second output signal, the second frequency band associated with clonic phase seizure activation of muscle to provide a second output signal.
26. The system of clause 25 further comprising one or more sensor units, at least one of the one or more sensor units including said one or more electrodes.
27. The system of clause 26, the at least one sensor units including each of said first analysis module and said second analysis module.
28. The system of clause 25 further comprising a time-point extraction module, the time-point extraction module configured to compare at least one of said first output signal and said second output signal to at least one threshold value and to determine a starting point of one or more phases of a detected seizure event based on meeting the at least one threshold value.
29. The system of clause 25, at least one of said one or more processor units configured for recording at least a portion of the received electrical signal.
30. The system of clause 27, each of said first analysis module, said second analysis module and said time-point extraction module being part of a common processor unit.
31. The system of clause 25, each of said first analysis module and said second analysis module being part of a common processor unit;
   said common processor unit further configured to provide each of said first output signal and said second output signal to a second processor unit, the second processor including a time-point-extraction module.
32. The system of clause 25, said one or more processor units comprising a base station.
33. The system of clause 32, said base station including said time-point extraction module, the base station configured to provide a report indicating the semiology of a detected seizure to one or more caregiver devices.
34. The system of clause 32, said base station including said time-point extraction module, the base station configured to provide an automatically generated report indicating the semiology of a detected seizure to one or more caregiver devices.
35. The system of clause 25, at least one of said one or more processor units being in wireless communication with a sensor unit, the sensor unit including said one or more electrodes.
36. The system of clause 25, further comprising one or more filters for filtering the received signal in order to isolate said second frequency band, the second frequency band excluding at least a portion of the delta band, the theta band, or both.
37. The system of clause 25 further comprising one or more filters for filtering the received signal in order to isolate said second frequency band, the second frequency band including a low frequency boundary of about 3.5 Hz.
38. The system of clause 25 further comprising one or more filters for filtering the received signal in order to isolate said second frequency band, the second frequency band including a low frequency boundary of about 6 Hz.
39. The system of clause 25, said first frequency band including one or more frequencies within a range of frequencies from about 32 Hz to about 512 Hz.
40. The system of clause 25, said second frequency band including one or more frequencies within a range from about 2 Hz to about 16 Hz.
41. The system of clause 25, said second frequency band excluding at least a portion of the delta band above about 2 Hz, the theta band, or both.
42. The system of clause 25 further comprising an evaluation module, the evaluation module configured to calculate a ratio (HF/LF) between said first output signal and said second output signal and to generate an alarm signal if the ratio (HF/LF) meets a threshold ratio for seizure detection.
43. The system of clause 25 further comprising an evaluation module, the evaluation module configured to calculate a ratio (HF/LF) between said first output signal and said second output signal and to generate an alarm signal if the ratio (HF/LF) meets a threshold ratio for seizure detection and one or more of said first output signal and second output signals meets a threshold value.
44. The system of clause 27 wherein said time-point extraction module is further configured to apply a zero-crossing function to one or more of the first output signal or the second output signal and to count the number of crossings within one or more time windows.
45. The system of clause 25 further comprising said one or more electrodes, the electrode configured for positioning at one or more of the F7, F8, T3, and T4 positions.
46. The system of clause 25 wherein said one or more electrodes are part of an EEG headset, the EEG headset further including one or more additional electrodes configured to collect an electrical signal indicative of patient brain activity.
47. A method of analyzing the dynamics of a seizure, the method comprising:
   collecting a signal from one or more electrodes positioned on the head or scalp of a patient;
   filtering the collected signal to provide a first frequency band associated with muscle-related electrical activity elevated during a patient seizure;
   filtering the collected signal to provide a second frequency band associated with muscle-related electrical activity elevated during a clonic-phase of a patient seizure;
   processing the signal in one or more of said first frequency band and said second frequency band to determine a starting point and an ending point of a seizure or seizure-related event; and
   using the starting point and an ending point of said seizure or seizure-related event to determine the dynamics of said seizure or seizure-related event.
48. The method of clause 47, wherein said one or more electrodes are part of a wireless EEG-headset.
49. The method of clause 47, wherein said one or more electrodes are positioning at one or more of the F7, F8, T3, and T4 positions.
50. The method of clause 47 wherein said second frequency band ranges from about 3.5 Hz to about 16 Hz.
51. The method of clause 47 further comprising:
   determining a power content in said second frequency band; and
   comparing said power content in said second frequency band to a threshold value in order to determine a starting point for a clonic-phase portion of said seizure.
52. The method of clause 51 wherein said threshold value is determined based on a power of collected signal in said second frequency band at a time of detection of said seizure.
53. The method of clause 51 wherein said threshold value is determined based on a strength of collected signal measured at a time when said seizure was detected based on a ratio (HF/LF) of signals determined from said first frequency band and said second frequency band.
54. The method of clause 47 wherein said first frequency band ranges from about 32 Hz to about 512 Hz.
55. The method of clause 47 further comprising:
   comparing the power content in said first frequency band to a threshold value in order to determine a starting point for said seizure.
56. The method of clause 47 further comprising:
   determining a number of zero-crossings of a transformed signal of the first frequency band and comparing said number of zero-crossings to a threshold level in order to determine a starting point for said seizure.
57. The method of clause 47 further comprising:
   determining a number of zero-crossings of a transformed signal of the first frequency band and comparing said number of zero-crossings to a threshold level in order to determine an end point for said seizure.

Although the disclosed subject matter and its advantages have been described in detail, it should be understood that various changes, substitutions and alterations can be made herein without departing from the invention as defined by the appended claims. Moreover, the scope of the claimed subject matter is not intended to be limited to the particular embodiments of the process, machine, manufacture, composition, or matter, means, methods and steps described in the specification. Among other things, any feature described for one embodiment may be used in any other embodiment, and methods described and shown in the figures may be combined. In addition, the order of steps shown in the figures and described above may be changed in different embodiments. Use of the word “include,” for example, should be interpreted as the word “comprising” would be, i.e., as open-ended As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods or steps.

What is claimed is:

1. A system for determining the dynamics of seizures, the system comprising:
one or more processor units capable of receiving an electrical signal, the electrical signal being collected from one or more electrodes disposed on the head or scalp of a patient, the one or more processor units including;
a first analysis module configured to analyze said electrical signal in a first frequency band within a range from about 32 Hz to about 512 Hz to provide a first output signal;
a second analysis module configured to analyze said electrical signal in a second frequency band within a range from about 2 Hz to about 16 Hz to provide a second output signal; and
a time-point extraction module configured to compare at least one of the first output signal or said second output signal to at least one threshold value and to determine a starting point of at least one seizure event phase based on meeting the at least one threshold value.

2-4. (canceled)

5. The system of claim 1 further comprising a sensor unit, the sensor unit including said one or more electrodes.

6-13. (canceled)

14. The system of claim 1 further comprising one or more filters for filtering said received signal in order to isolate said second frequency band, the second frequency band including a low frequency boundary of about 3.5 Hz.

15. The system of claim 1 further comprising one or more filters for filtering said received signal in order to isolate said second frequency band, the second frequency band including a low frequency boundary of about 6 Hz.

16. The system of claim 1 further comprising an evaluation module, the evaluation module configured to generate an alarm signal in response to seizure detection.

17. The system of claim 1 further comprising an evaluation module, the evaluation module configured to calculate a ratio (HF/LF) between said first output signal and said second output signal and to generate an alarm signal if the ratio (HF/LF) meets a threshold ratio for seizure detection.

18-20. (canceled)

21. The system of claim 5 the one or more electrodes configured for positioning at one or more of the F7, F8, T3, and T4 positions.

22. (canceled)

23. The system of claim 1, said one or more electrodes included as part of an EEG headset, the EEG headset further including one or more additional electrodes configured to collect an electrical signal indicative of patient brain activity.

24. (canceled)

25. A system for monitoring a patient for seizure activity, the system comprising:
one or more sensor units, at least one of the one or more sensor units including one or more electrodes, the one or more electrodes configured to be disposed on the head or scalp of a patient;
a processor unit capable of receiving an electrical signal collected from said one or more electrodes, the processor unit configured to analyze the received electrical signal for detection of seizure events based on muscle-related electrical activity, the processor unit including;
a first analysis module configured to analyze the received electrical signal within a first frequency band to provide a first output signal, the first frequency band associated with tonic-clonic seizure activation of muscle; and
a second analysis module configured to analyze the received signal within a second frequency band to provide a second output signal, the second frequency band associated with clonic phase seizure activation of muscle to provide a second output signal.

26. (canceled)

27. (canceled)

28. The system of claim 25 further comprising a time-point extraction module, the time-point extraction module configured to compare at least one of said first output signal and said second output signal to at least one threshold value and to determine a starting point of one or more phases of a detected seizure event based on meeting the at least one threshold value.

29-40. (canceled)

41. The system of claim 25, said second frequency band excluding at least a portion of the delta band above about 2 Hz, the theta band, or both.

42-46. (canceled)

47. A method of analyzing the dynamics of a seizure, the method comprising:
collecting a signal from one or more electrodes positioned on the head or scalp of a patient;
filtering the collected signal to provide a first frequency band associated with muscle-related electrical activity elevated during a patient seizure;
filtering the collected signal to provide a second frequency band associated with muscle-related electrical activity elevated during a clonic-phase of a patient seizure;
processing the signal in one or more of said first frequency band and said second frequency band to determine a starting point and an ending point of a seizure or seizure-related event; and
using the starting point and an ending point of said seizure or seizure-related event to determine the dynamics of said seizure or seizure-related event.

48. The method of claim 47, wherein said one or more electrodes are part of a wireless EEG-headset.

49. The method of claim 47, wherein said one or more electrodes are positioning positioned at one or more of the F7, F8, T3, and T4 positions.

50. The method of claim 47 wherein said second frequency band ranges from about 3.5 Hz to about 16 Hz.

51. The method of claim 47 further comprising:
determining a power content in said second frequency band; and
comparing said power content in said second frequency band to a threshold value in order to determine a starting point for a clonic-phase portion of said seizure.

52. The method of claim 51 wherein said threshold value is determined based on a power of collected signal in said second frequency band at a time of detection of said seizure.

53. The method of claim 51 wherein said threshold value is determined based on a strength of collected signal measured at a time when said seizure was detected based on a ratio (HF/LF) of signals determined from said first frequency band and said second frequency band.

54. The method of claim 47 wherein said first frequency band ranges from about 32 Hz to about 512 Hz.

55. The method of claim 47 further comprising:
comparing the power content in said first frequency band to a threshold value in order to determine a starting point for said seizure.

56. (canceled)

57. (canceled)
